SEQUENCE LISTING (37 sequences)

SEQ ID NO: 1 - DNA, 25 bp, Artificial Sequence
  Primer
caaccaacaagtgatattctccatg

SEQ ID NO: 2 - DNA, 21 bp, Artificial Sequence
  Primer
gatccacactctccagctgca

SEQ ID NO: 3 - DNA, 25 bp, Artificial Sequence
  Primer
tgatggacctacaggagctcctgag

SEQ ID NO: 4 - DNA, 25 bp, Artificial Sequence
  Primer
gagtcaaatccagaacatgccgcag

SEQ ID NO: 5 - DNA, 30 bp, Artificial Sequence
  Primer
acgaggtcacaggagaagggacgccatgca

SEQ ID NO: 6 - DNA, 29 bp, Artificial Sequence
  Primer
tcttcatggagcagcttatcgatgaatcc

SEQ ID NO: 7 - DNA, 24 bp, Artificial Sequence
  Primer
gtgacaaccacggccttccctact

SEQ ID NO: 8 - DNA, 18 bp, Artificial Sequence
  Primer
ggtagctatggtactcca

SEQ ID NO: 9 - DNA, 19 bp, Artificial Sequence
  Primer
tgttgtagaggtggactgg

SEQ ID NO: 10 - DNA, 20 bp, Artificial Sequence
  Primer
tggcaggacactgaatactt

SEQ ID NO: 11 - DNA, 24 bp, Artificial Sequence
  Primer
tggaggaactggcaaaaggatggt

SEQ ID NO: 12 - DNA, 21 bp, Artificial Sequence
  Primer
ttgggacaatctcttccccac

SEQ ID NO: 13 - DNA, 21 bp, Artificial Sequence
  Primer
gcgacgtggaactggcagaag

SEQ ID NO: 14 - DNA, 20 bp, Artificial Sequence
  Primer
gccttgagcagttcaccttc

SEQ ID NO: 15 - DNA, 20 bp, Artificial Sequence
  Primer
ctcttctacggggacagcag

SEQ ID NO: 16 - DNA, 20 bp, Artificial Sequence
  Primer
acttttggacaccaggttgg

SEQ ID NO: 17 - DNA, 20 bp, Artificial Sequence
  Primer
agccacaatccagtcattcc

SEQ ID NO: 18 - DNA, 23 bp, Artificial Sequence
  Primer
gaatgctgatggtcgttttatta

SEQ ID NO: 19 - DNA, 20 bp, Artificial Sequence
  Primer
gacggcactcttgctaggct

SEQ ID NO: 20 - DNA, 20 bp, Artificial Sequence
  Primer
tgcctgcctgggatctgtaa

SEQ ID NO: 21 - DNA, 20 bp, Artificial Sequence
  Primer
ggacgaggcaagagtttcac

SEQ ID NO: 22 - DNA, 20 bp, Artificial Sequence
  Primer
acctggaccacaacaaggtc

SEQ ID NO: 23 - DNA, 20 bp, Artificial Sequence
  Primer
caccttctccaggttggtgt

SEQ ID NO: 24 - DNA, 22 bp, Artificial Sequence
  Primer
gaacatcacctaccactgcaag

SEQ ID NO: 25 - DNA, 22 bp, Artificial Sequence
  Primer
gcagagtcctagagtgactgag

SEQ ID NO: 26 - DNA, 20 bp, Artificial Sequence
  Primer
aatcccatcaccatcttcca

SEQ ID NO: 27 - DNA, 20 bp, Artificial Sequence
  Primer
tgtggtcatgagtccttcca

SEQ ID NO: 28 - DNA, 20 bp, Artificial Sequence
  Primer
acccagtgaggccttattcc

SEQ ID NO: 29 - DNA, 20 bp, Artificial Sequence
  Primer
tgatcactgcaggaaactgg

SEQ ID NO: 30 - DNA, 21 bp, Artificial Sequence
  Primer
atgaactgtgtttgccgcctg

SEQ ID NO: 31 - DNA, 21 bp, Artificial Sequence
  Primer
gagctgtagagctcccagtgc

SEQ ID NO: 32 - DNA, 20 bp, Artificial Sequence
  Primer
ctcggcctttgtgtttccag

SEQ ID NO: 33 - DNA, 20 bp, Artificial Sequence
  Primer
tggcaggagatgccaaagat

SEQ ID NO: 34 - DNA, 20 bp, Artificial Sequence
  Primer
ttcagaaatgccttgcagtg

SEQ ID NO: 35 - DNA, 20 bp, Artificial Sequence
  Primer
cacctctttgctctgctcct

SEQ ID NO: 36 - DNA, 20 bp, Artificial Sequence
  Primer
atgaggggaccacatctacg

SEQ ID NO: 37 - DNA, 20 bp, Artificial Sequence
  Primer
atctccagcctgtcaaatgg

(19) United States
(12) Patent Application Publication
(10) Pub. No.: US 2011/0052622 A1
(43) Pub. Date: Mar. 3, 2011

(54) REISHI-MEDIATED ENHANCEMENT OF HUMAN TISSUE PROGENITOR CELL ADHESION AND DIFFERENTIATION

(76) Inventors: Chi-Huey Wong, La Jolla, CA (US); Daniel Tzu-Bi Shih, Lake Oswego, OR (US); Wan-Yu Chen, Zhonghe City (TW)

(21) Appl. No.: 12/547,388

(22) Filed: Aug. 25, 2009

(51) Int. Cl. A61K 36/06

(52) U.S. Cl. 424/195.15

(57) ABSTRACT

The present disclosure provides medicinally active extracts and fractions, and methods for using the same to increase eukaryotic cell adhesion, to increase differentiation of eukaryotic cells to produce increased numbers of immature dendritic cells, to enhance or accelerate condrogenesis of MSCs, to maintain undifferentiated CD34+ hematopoietic stem cells, and to inhibit or reduce the blood EPCs formation and angiogenic potential. These methods are useful for modulating immune response, modulating hematopoietic activity, inhibiting tumor angiogenesis, and engineering certain types of eukaryotic tissues.

### RELATED APPLICATION

This disclosure is a continuation-in-part and claims the Paris Convention priority of U.S. Utility application Ser. No. 11/859,738, filed Sep. 21, 2007, which is incorporated by reference herein in its entirety, and incorporates by reference U.S. Provisional Application No. 60/846,676, filed Sep. 21, 2006.

### BACKGROUND

Somatic tissue stem/progenitor cells are preserved for replenishing the damaged and senescent tissue cells and for maintaining the homeostasis of healthy tissues due to their regenerative potential. Molecules that affect tissue stem/progenitor cells may thus have implications in regenerative medicine, aging, and cancer therapy. For example, oxysterols have pro-bone and anti-fat functions and Oncostatin M activates mesenchymal stem cells (MSCs) not only for self renewal but also for promoting bone formation and suppressing adipogenic differentiation. Recent investigation of the protease inhibitor Bortezomib (Bzb) has also shown that it enhances bone regeneration by targeting MSCs and has been used to treat bone marrow cancer to improve bone density in a mouse model.

Cell adhesion molecules (“CAMs”) play a key role in mobilizing cell-cell interactions, particularly during embryonic development, neural plasticity, and tumor metastasis. Glycoside conjugates on cellular membranes play an important role in determination of stem cell and stem cell-derived eukaryotic cell homing, mobilization, differentiation, morphogenesis, adhesion, and have transforming properties. Thus, identifying and characterizing glycoconjugate and membrane receptor expression on normal tissue progenitor cells is critical in controlling the health, disease state, and homeostasis of eukaryotic cells and eukaryotic cell-based organisms.

Cooperative regulation of N-glycans in Golgi complex has been shown to influence the proliferation and differentiation activities of stem and progenitor cells. Glycoconjugates on the surface of neural stem cells (NSCs) have also been shown to have a wide range of functions associated with receptor mediated signaling. For example, basic fibroblast growth factor, an important mitogen of NSCs, requires heparan sulfates, proteoglycans, and glycosylated cystatin C for activity. Notch signaling, which regulates a wide variety of developmental processes, is modulated by a fucose containing glycan. In addition, normal tissue stem/progenitor cells and cancer cells frequently display glycans with different levels or different structures than differentiated tissue cells.

Most Cell adhesion molecules on cell surfaces contain glycoconjugates and affect development, aging, and disease states and have been shown to play a key role in modulating embryonic development, cell-cell interactions, neural plasticity, and tumor metastasis. For example, the content of N-CAM polysialic acid (polySia) changes dramatically during embryonic development and human breast cancer progression. Among CAMs, the immunoglobulin (Ig) super family regulates tissue development and receptor-induced intracellular signal cascades. For example, V-CAM expressed on bone marrow stromal cells is responsible for the binding of hematopoietic and angiogenic progenitor cells, and N-CAM (CD56) expression is important during neural development and hematopoiesis. A recent study of N-CAM suggested that expression of CAMs enhances pericyte-endothelial cell interactions and inhibits cancer metastasis. Changes in the glycosylation pattern of N-CAM affect high-order structures and influence the activities of neural cells. N-CAM deficiency or dis-regulation has been found in various cancer metastasis (Table 1).

TABLE 1
Types of N-CAM associated with tumor metastasis

| Tumor Type | N-CAM | V-CAM | I-CAM |
|---|---|---|---|
| Breast Cancer4: | DisReg. | | |
| | [CD56, C16] | | |
| Oral Cancer (SACC) | Dec | Inc | Inc |
| Lymph angiogenesis. | Dec | | |
| | [induces Inc VEGFs] | | |
| Ewing tumor | DisReg | | |
| Prostate cancer | Unknown | | |
| | [Inc in Neuron] | | |
| Neuroblastoma | Dec | | |
| Ovarian carcinoma | Lost | | |
| adenoid cystic | Dec | | |
| carcinoma cells. | | | |
| melanoma | Dec | | |
| rectal cancer- | Dec | | Inc |
| lung cancer | Inc ? | | |
| [small cell]. | | | |
| Bile duct Cancer. | positive | | |
| Colorectal tumor | Dec | | Inc |
| Glioma | Dec | | |
| [BT4C & BT4Cn] | | | |

Blood monoculear cells (MNCs) can be transformed into circulating endothelial progenitor cells (EPCs) along with down regulation of endoglin (CD 105), PECAM (CD31) and up regulation of Flt-1 and KDR cell surface molecules under the stimuli of angiogenic factors and chemokines. In normal human tissues, endoglin, a cell membrane glycoprotein, is weakly expressed on erytroid precursors and stromal cells, whereas it is strongly expressed on proliferating endothelial cells. Endoglin is thus an attractive therapeutic target and represents a powerful marker for quantitative analysis of angiogenesis, tumor progression, and metastasis.

The metastatic spread of cancer cells is initiated by lymph angiogenesis. The most extensively studied signaling system that promotes cancer metastasis involved the secreted lymphangiogenic proteins, VEGF-C and VEGF-D, and their cognate receptor on lymphatic endothelium, VEGFR-3. Recent studies have identified other signaling molecules that also promote lymph angiogenesis in vivo, including angiogenin, angiopoietin, HGF, FGF, PDGF, and IGF families of secreted proteins. Angiogenin is a tRNA-specific ribonuclease, which binds to actin on the surface of endothelial cells. Once bound, angiogenin is endocytosed and translocated to the nucleus, thereby promoting invasive endothelial cells for blood vessel formation. Angiogenin induces vascularization of normal and malignant tissues, and is also required for FGF-b to induce tumor cell proliferation.

Recently, the polysaccharide fraction from Ganoderma lucidum (F3) has been shown to enhance human myeloid and lymphoid cell activity and promote immune cell function and anti-tumor activity. In addition, F3 alters cell immunophenotypic expression and enhance CD56+ NK-cell cytotoxicity in cord blood. The effect of F3 on human hematopoietic and mesenchymal tissue stem/progenitor cells has also been studied in an attempt to gain a better insight into its role in human tissue health, disease progression, and aging.

The present disclosure provides medicinally active extracts and fractions, and methods of preparing the same, from components of Ganoderma lucidum and Ganoderma tsugae, otherwise known as “reishi.” These extracts and fractions have been found to be especially active in modulating immune response and in increasing hematopoietic activity.

### SUMMARY

In one implementation, a method is disclosed comprising administering purified reishi extract to a subject, wherein blood mononuclear cell (MNCs) expression of cell adhesion molecules (CAMs) or cell surface markers is increased by at least 1%.

According to implementations, the cell adhesion molecules include at least VCAM, ICAM, or NCAM.

According to implementations, the combination is further comprised of an amount of mononuclear cells.

According to implementations, the primitive CD34+ hematopoietic stem cells comprise one or more of the group consisting of CD133+, CXCR4+, and CD38− cell surface markers.

According to implementations, the combination is further comprised of an amount of MSC or PLA cells.

According to implementations, a combination is disclosed comprising an amount of skeleton forming agent; and an amount of reishi extract.

According to implementations, the combination is further comprised of a medical device.

According to implementations, the combination is implantable into an organism.

According to implementations, a method is disclosed comprising administering purified reishi extract to a subject in an amount sufficient to stimulate MNCs to influence the dendritic cell maturity.

According to implementations, purified reishi is co-administered to a subject with at least one cytokine selected from the group consisting of IL-4 and GM-CSF.

According to implementations, Ganoderma lucidum is administered to a subject in an amount sufficient to stimulate blood mononuclear cells, wherein the cell surface markers expression of immature dendritic cell markers is increased by at least 1%, in AIM-V culture condition.

According to implementations, the immature dendritic cell markers are selected from the group consisting of expressions of CD1a, CD40, CD80, and CD86 cell surface markers.

According to implementations, a method is disclosed comprising administering purified reishi extract to a subject, wherein the cell surface markers expression of mature dendritic cell markers is increased by at least 1%, in RPMI culture condition, as shown in FIG. 3D and FIG. 3E.

According to implementations, the mature dendritic cell markers are selected from the group consisting of CD83+ cell surface marker.

According to implementations, a method is disclosed comprising administering purified reishi extract to a subject in an amount sufficient to reduce or inhibit angiogenesis of the blood mononuclear cells (MNCs) derived endothelium progenitor cells (EPCs).

According to implementations, Ganoderma lucidum is administered to a subject in an amount sufficient to reduce or inhibit angiogenesis.

According to implementations, Ganoderma lucidum reduces or inhibits the EPCs colony formation.

According to implementations, Ganoderma lucidum reduces or inhibits the expressions of angiogenic factors. According to implementations, reduction or inhibition of the expression of angiogenic factors includes at least the factors angiogenin, FLt-1, Flt-3, and VEGF in EPCs.

According to implementations, Ganoderma lucidum also reduces or inhibits the endothelial expressions of endoglin.

According to implementations, a method is disclosed comprising administering purified reishi extract to a subject, wherein the cell surface markers expression of primitive CD34+ hematopoietic stem cell (Primitive HSCs) is increased by at least 1%.

According to implementations, Ganoderma lucidum is administered to a subject in an amount sufficient to reduce or prevent differentiation of the primitive HSCs when the primitive HSCs are in presence of soluble growth factors and cytokines. According to implementations, such soluble growth factors and cytokines include at least one of IL-6, TPO, Flt-3, SCF, IL-3, or IGF.

According to implementations, the primitive HSCs surface markers are selected from the group consisting of CD45 hematopoietic cell markers.

According to implementations, the group of the primitive HSCs are selected from the group consisting of CD38−, CD133+, CXCR4+, or Lin-cell surface markers.

According to implementations, a method is disclosed comprising administering purified reishi extract to a subject in an amount sufficient to accelerate or enhance chondrosphere formation of MSCs or PLA by at least 1%.

According to implementations, a combination is disclosed comprising an amount of skeleton forming agent, and an amount of reishi extract.

According to implementations, Ganoderma lucidum is co-administered to a subject with at least one compound selected from the group consisting of insulin, TGF-B1, or ascorbate-2-phosphate.

According to implementations a method is disclosed comprising administering purified reishi extract to a subject, wherein MSC or PLA expression of cellular proteins is increased by at least 1%.

According to implementations, Ganoderma lucidum increases expression of one or more of the cellular proteins comprising condrogenic factors including N-CAM, ICAM, IL-11, BMP-2, and aggrecan.

### DRAWINGS

The above-mentioned features and objects of the present disclosure will become more apparent with reference to the following description taken in conjunction with the accompanying drawings wherein like reference numerals denote like elements and in which:

FIG. 1 depicts results of implementations of experimental data showing the percentage of suspended and adherent mononuclear cells expressing particular cell surface markers in vitro when incubated with and without purified reishi, as characterized by FACS analysis;

FIGS. 2A-2E are results of implementations of experimental data showing the effect of reishi on mononuclear cells;

FIGS. 3A-3G depict the relative level of expression of V-CAM and N-CAM cell surface markers expressed by cells in vitro; FIG. 3A shows results with Western Not analysis; The axes on FIG. 3A are as follows: Horizontal (left to right): CD1a(+)/CD14(−); CD40, CD80; CD83; CD86; Vertical: CD marker expression %; FIGS. 3B and 3C depict the percentage of cells expressing particular cell surface markers in vitro when incubated with and without purified reishi, as characterized by FACS analysis; the axes on FIG. 3B are as follows: Horizontal (left to right): CD1a(+)/CD14(−); CD40; CD80; CD83; CD86; Vertical: CD marker expression %; FIG. 3D depicts the relative population of cells expressing the CD19+ cell surface marker in vitro when incubated with and without purified reishi, as characterized by FACS analysis; the axes on FIG. 3D are as follows: Horizontal (left to right): CD1a; CD3; CD14; CD16, CD19; CD34+CD45; CD56; CD83; Vertical: ratio to the control. 0; 0.5; 1; 1.5; 2; 2.5; 3; 3.5; FIG. 3E depicts the relative population of cells expressing the CD83 cell surface market in vitro when incubated with and without purified reishi in RPMI culturing condition, as characterized by FACS analysis; the axes on FIG. 3E are as follows: Horizontal: CD1a; CD3; CD14; CD19; CD56; CD83; Vertical: ratio to contr. 0; 0.5; 1; 1.5; 2; 2.5; 3; 3.5; FIGS. 3F and 3G depict the relative levels of various cytokines expressed by cells incubated in vitro with and without reishi extract, as characterized by mRNA (cDNA, PCR) analysis; the axes on FIG. 3F are as follows: Horizontal: IL-1; IL-6; MCP-1; RANTES; GRO; GRO-1; MIP-1; Vertical: 0; 20; 40; 60; 80; 100. The axes on FIG. 3G are as follows: Horizontal: angiogenin; IL-4; PARC; Vertical: 0; 20; 40; 60; 80; 100;

FIGS. 4A and 4B depict the relative number of HSCs incubated in vitro with and without reishi extract that express the CD34+ stem/progenitor cell surface marker and the primitive CD34+/CD38− hematopoietic stem cell population, assessed by FACS; The axes on FIG. 4A are as follows: Vertical: 0; 5; 10; 15; 20; 25; Horizontal: DAY1; DAY4; DAY7; DAY12; the axes on FIG. 4B are as follows: Vertical: 0%; 20%; 40%; 60%; 80%; 100%; Horizontal: CD34+; CD34+/CD38−; CD34+/CD38+; 38+; 38−; FIGS. 4C-4F depict the relative number of cells incubated in vitro with and without various reishi extract fractions that express the CD34+ cell surface marker and the CD34+/CD38− proteome, assessed by FACS or total mRNA analysis; the axes on FIG. 4C are as follows: Vertical: “ratio to control” 0, 1, 2, 3; Horizontal: F3(100 μg/ml); F6-10 (10 μg/ml); F11-15 (10 μg/ml); F16-20 (10 μg/ml); the axes on FIG. 4D are as follows: Vertical: “ratio to control” 0, 0.5, 1, 1.5, 2, 2.5, 3; Horizontal: F3(100 μg/ml); F6-10 (10 μg/ml); F11-15 (10 μg/ml); F16-20 (10 μg/ml); the axes on FIG. 4E are as follows: Vertical: “ratio to control” 0, 0.5, 1, 1.5, 2, 2.5, 3; Horizontal: F3(100 μg/ml); F6-10 (10 μg/ml); F11-15 (10 μg/ml); F16-20 (10 μg/ml). The axes on FIG. 4F are as follows: Vertical: “ratio to control” 0, 2,4, 6,8; Horizontal: F3(100 μg/ml); F6-10 (10 μg/ml); F11-15 (10 μg/ml); F16-20 (10 μg/ml);

FIGS. 5A-5C depict the relative number of mesenchymal stem cells incubated with and without reishi extract in vitro that differentiate to chondroblasts, characterized by chondrosphere formation (via phase-contrast microscopy), RT-PCR analysis of various cellular gene transcriptions on MSC in condrogenesis; the axes in 5A; are as follows: Vertical—“condrocyte (SIC) number” 0, 50, 100, 150; Horizontal—day 0, day 3, day 7; the legends are: control, F3 (50 μg/ml), F3(100 μg/ml); the axes in 5B are as follows: Vertical—“Chondrocyte” 0; 200; 400; 600; 800; 1000; 1200; 1400; 1600; Horizontal—day 3; day 7. The Legends are: control, +F3 (100 μg/ml);

FIGS. 6A-6C depict the relative number of mesenchymal stem cells incubated with and without reishi extract in vitro that differentiate to chondroblasts, characterized by chondrosphere formation (via phase-contrast microscopy), RT-PCR analysis of various cellular gene transcriptions on MSC in condrogenesis according to of implementations of experimental data; in the graph of FIG. 6B: Vertical column—“condrocyte (SIC) number” 0, 50, 100, 150; Horizontal column—day 0; day 3; day 7; legend: —control, −F3 (50 μg/ml), −F3 (100 μg/ml); in the left Western blot shown in FIG. 6C: Vertical—Cbfa-1, BMP-2, BMP-4, PPAR-r, IL-11; Horizontal—CM only; CM+F3, in right hand Western blot of FIG. 6C: Vertical—Aggregan, Collagen type II, chondroadherin, NCAM, ICAM, GAPDH; Horizontal—CM only, CM+F3;

FIGS. 7A-7D are results of implementations of experimental data showing F3's effect on HSC expansion in culture, as previously shown in FIGS. 3A-3G, and FIGS. 4A-4F; FIG. 7A: F3 effect on HSC expansion in culture, F3 maintains the primitive HSCs in a cytokine cocktail culture, as shown by the influence of F3 (10 μg/ml) (F3+) on CD34+ HSCs (by fold) in an ex vivo expansion culture; FIGS. 7B-7D: F3 exhibits TPO like function in the HSC expansion culture, as shown in a cytokine replacement experiment; the cocktail for the ex vivo HSC expansion system consists of SCF, Flt-3L, TPO, IL-6 and IL-3 cytokines in IMDM medium; F3 was found equivalent to TPO in maintenance of CD38−, AC133+, and CXCR4+ primitive HSC subpopulations, in the expansion culture; (*, P<0.05; **, P<001; ***, P<0.001);

FIGS. 8A-8C are results of implementations of experimental data depicting how F3 affects blood dendritic cell (DC) formation in culture, as previously shown in FIG. 2A. FIG. 8A showed that F3 significantly increased the attachment of MNCs, and induced morphology changes in the culture, regardless of the presence of GM-CSF or IL-4 in AIM-V culture media; the transcriptional gene expression of NCAM and VCAM FIG. 8B and immature surface markers (CD1a, CD40, CD80) was increased by F3 treatment FIG. 8C in AIM-V medium, supplemented with GM-CSF (GM) alone or IL-4 (+4) alone or GM-CSF and IL-4 together (+G+4);

FIGS. 9A-9B are results of implementations of experimental data depicting the F3 effect on human blood MNC cytokine expression. F3 caused a significant decrease in the secretion of angiogenin and PARC and an increase in the levels of IL-6, GRO, MIP-1, MCP-1, and RANTES in blood MNCs; and

FIGS. 10A-10D are results of implantations of experiments depicting the F3 effect on angiogenesis; in FIG. 10A, F3 reduces the blood EPCs colony formation in culture, but does not affect the Ad-MSCs derived EC tube forming activities in FIG. 10B; F3 inhibits, dose dependently, the expression of endoglin (CD105) as analyzed by FACS, as shown in FIG. 10C. In FIG. 10D, F3 was shown to up regulate IL-10, ENA-8, and GRO and down regulate Eotaxin-2, IL-13, IL-1ra, MCP-2, MCD, MIP-2, TNF-α, and ICAM-1 secretions.

### DETAILED DESCRIPTION

In the following detailed description of embodiments of the present disclosure, reference is made to the accompanying drawings in which like references indicate similar elements, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims. As used in the present disclosure, the term “or” shall be understood to be defined as a logical disjunction and shall not indicate an exclusive disjunction unless expressly indicated as such or notated as “xor.”

For the purposes of describing the present disclosure, the following terms are intended to refer to the associated definitions as described below:

“Angiogenesis” means a physiological process involving the growth of new blood vessels from pre-existing vessels.

“Adherent Cells” means cells that remain attached to the sides of an incubation vessel subsequent to aspiration of incubation media. Adherent cells may be subsequently dissociated from the sides of an incubation vessel by means of chemical or physical processes, e.g., through application of a trypsin solution.

“Administering” means oral, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection and infusion as well as co-administration as a component of any medical device or object to be inserted (temporarily or permanently) into the body.

“Aggrecan” means a proteoglycan, or a protein modified with carbohydrates. Along with collagen, aggrecan forms a major structural component of cartilage, particularly articular cartilage.

“Amount of cells adhering” means an assay of the relative number of cells adhering to the surfaces of an incubation vessel. This assay may be carried out in a variety of ways, such as through visual observation of the degree of confluence of adherent cells on the surfaces of an incubation vessel, or through trypsinization of the adherent cells and subsequent counting of a representative sample of the trypsinized cells in solution.

“B cell” means any of a class of lymphocytes that play a large role in the humoral immune response as opposed to the cell-mediated immune response that is governed by T cells. B cells are produced in the bone marrow of most mammals and are therefore called B cells. The principal function of B cells is to make antibodies against soluble antigens. B cells are an essential component of the adaptive immune system.

“BMP-2” means bone morphogenic protein, a type of cytokine.

“Buffer Solution” means a solution which resists change in hydrogen ion and hydroxide ion concentration (and consequently pH) upon addition of small amounts of acid or base, or upon dilution. Buffer solutions consist of a weak acid and its conjugate base (more common) or a weak base and its conjugate acid (less common).

“Cartilage” means a tough, elastic, fibrous connective tissue found in various parts of the body, such as the joints, outer ear, and larynx. A major constituent of the embryonic and young vertebrate skeleton, it is converted largely to bone with maturation.

“CD” means cluster of differentiation, a protocol used for the identification and investigation of cell surface molecules present on leukocytes. CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signalling, but have other functions, such as cell adhesion.

“Cell Surface Markers” means a protein that is present in the cell surface of a eukaryotic cell, as well as any gene expression product specific to that particular protein (whether characterized in vivo or in vitro)(for example, mRNA or cDNA).

“Centrifuge” means an apparatus consisting essentially of a compartment spun about a central axis to separate contained materials of different specific gravities, or to separate colloidal particles suspended in a liquid.

“Chondrocyte” means a type of eukaryotic cell found in cartilage. Chondrocytes produce and maintain the cartilaginous matrix, which consists mainly of collagen and proteoglycans. The progenitors of chondrocytes are mesenchymal stem cells.

“Chondrogenesis” means the process by which cartilage is formed.

“Cytokine” means any of a group of proteins and peptides that are used in organisms as signaling compounds. These chemical signals are similar to hormones and neurotransmitters and are used to allow one cell to communicate with another.

“Dendritic Cells” means immune cells that form part of the mammalian immune system. Their main function is to process antigen material and present it on the surface to other cells of the immune system, thus functioning as antigen-presenting cells.

“Dendritic Cell Markers” means any of a group of cell surface molecules found generally on the surface of dendritic cells.

“Differentiate” means the process by which eukaryotic cells acquire a “type” (e.g., dendritic cell, chondrocyte); e.g., a change in cellular morphology without a requirement of a change in genetic material.

“Endothelial” means the thin layer of cells that line the interior surface of blood vessels, forming an interface between circulating blood in the lumen and the rest of the vessel wall.

“Expression” means the process by which inheritable information which comprises a gene, such as the DNA sequence, is made manifest as a physical and biologically functional gene product, such as protein or RNA. Expression may be quantitated by immunological (e.g., MACS, FACS) and/or by molecular biology (e.g., total RNA analysis) techniques.

“FACS” means Fluorescence Activated Cell Sorting (e.g., flow cytometry). FACS is a powerful method used to study and purify cells. Individual cells held in a thin stream of fluid are passed through one or more laser beams cause light to scatter and fluorescent dyes to emit light at various frequencies. Photomultiplier tubes (PMT) convert light to electrical signals and cell data is collected. Cell sub-populations are identified and sorted at high purity (˜100%) based upon the charge of a fluorescent dye linked to a particular cell type via an antibody-antigen relationship.

“Ficoll-Hypaque” means a density-gradient centrifugation technique for separating lymphocytes from other formed elements in the blood; the sample is layered onto a Ficoll-sodium metrizoate gradient of specific density; following centrifugation, lymphocytes are collected from the plasma-Ficoll interface.

“Gel filtration” means separation of proteins, peptides, and oligonucleotides on the basis of size. Molecules move through a bed of porous beads, diffusing into the beads to greater or lesser degrees. Smaller molecules diffuse further into the pores of the beads and therefore move through the bed more slowly, while larger molecules enter less or not at all and thus move through the bed more quickly. Both molecular weight and three dimensional shape contribute to the degree of retention. Gel Filtration Chromatography may be used for analysis of molecular size, for separations of components in a mixture, or for salt removal or buffer exchange from a preparation of macromolecules.

“Glycoconjugate” means a type of compound comprising carbohydrate units covalently linked with other types of chemical constituent.

“Glycoprotein” means proteins that contain oligosaccharide chains (glycans) covalently attached to their polypeptide backbones.

“Glycoside” means certain molecules in which a sugar part of the molecule is bound to some other part of the molecule.

“Glycosylation” means the process or result of addition of saccharides to proteins and lipids.

“GM-CSF” means Granulocyte-macrophage colony-stimulating factor, a cytokine.

“Hematopoietic Cells” means blood forming stem cells. T cells and B cells, among other cell types, arise from these cells.

“Hematopoietic Lineage Markers” means any of a group of cell surface molecules found generally on the surface of hematopoietic stem cells.

“Immature Dendritic Cells” means dendritic cells characterized by high endocytic activity and low T-cell activation potential. Immature dendritic cells are capable of phagocytosing pathogens and other sources of protein.

“Increased Expression of Cell Markers” means an increased quantity of a particular type of cell surface protein or mRNA molecule coding for that particular type of cell surface protein. Various protein molecules are associated with particular eukaryotic cell morphologies. Increased expression may be assayed using antibody-linked cell sorting (“FACS”) or through magnetic-activated cell sorting (“MACS”). Alternately, increased expression may be assayed by use of RT-PCR, in which the quantity of intracellular expression of mRNA coding for production of a particular type of CD molecule is indirectly determined.

“Incubate” means to grow or maintain eukaryotic cells in vitro in a vessel (optionally, plastic or glass) and in a liquid medium at conditions of approximately 37° Celsius, 5% carbon dioxide and some degree of humidity. The incubated eukaryotic cells are optionally supplemented with any combination of growth media, cytokines or other buffering or other solutions.

“Interleukin” means any of a group of cytokines (secreted signaling molecules) that were first seen to be expressed by white blood cells (leukocytes, hence the -leukin) as a means of communication (inter-). The function of the immune system depends in a large part on interleukins, and rare deficiencies of a number of them have been described, all featuring autoimmune diseases or immune deficiency. Interleukins are commonly designated using an abbreviation: e.g., IL-1, IL-2, etc.

“Lose Expression” means the process by which a particular expressed gene (manifest, e.g., in a particular protein or mRNA sequence) is no longer present or is expressed in reduced quantities or frequencies in a eukaryotic cell. Loss of expression is quantifiable via MACS or FACS analysis or via total mRNA analysis.

“Lyophilize” means a freeze-drying dehydration process typically used to preserve a perishable material or make the material more convenient for transport. Freeze drying works by freezing the material and then reducing the surrounding pressure and adding enough heat to allow the frozen water in the material to sublime directly from the solid phase to gas.

“Macrophage” means cells within the tissues that originate from specific white blood cells called monocytes. Monocytes and macrophages are phagocytes, acting in both nonspecific defense (or innate immunity) as well as specific defense (or cell-mediated immunity) of vertebrate animals. Their role is to phagocytose (engulf and then digest) cellular debris and pathogens either as stationary or mobile cells, and to stimulate lymphocytes and other immune cells to respond to the pathogen.

“MACS” means Magnetically Activated Cell Sorting. MACS is a separation technique that isolates rare cells from whole blood by binding the cells to anti-body labeled paramagnetic beads. The blood is passed through a column with a high magnetic field gradient that traps the paramagnetic beads.

“Mature Dendritic Cells” means dendritic cells that have come into contact with a pathogen and are capable of presenting pathogen protein fragments at their cell surfaces.

“Mesenchyme” (also known as embryonic connective tissue) means the mass of tissue that develops mainly from the mesoderm (the middle layer of the trilaminar germ disc) of an embryo. Viscous in consistency, mesenchyme contains collagen bundles and fibroblasts. Mesenchyme later differentiates into blood vessels, blood-related organs, and connective tissues.

“Mononuclear cells” means large, phagocytic mononuclear leukocytes produced in the vertebrate bone marrow and released into the blood and tissues where they develop into myeloidal white blood cells such as monocytic macrophages; contain a large, oval or somewhat indented nucleus and surrounded by voluminous cytoplasm and numerous organelles. For purposes of describing the present disclosure, mononuclear cells includes mononuclear cells in vitro, in vivo and in vivo but subsequently isolated and extracted.

“Morphology” means the outward appearance (shape, structure, color, pattern) or identity of a eucaryotic cell, organism, or organism component.

“mRNA” means Messenger Ribonucleic Acid. mRNA is a molecule of RNA encoding a chemical “blueprint” for a protein product.

“MSC” means a type of mesenchymal connect tissue stem/stromal cells, bone marrow-derived mesenchymal stem/stromal cell as an example of MSCs.

“NCAM” means a cell surface molecule important in mediating binding of eukaryotic cells with other eukaryotic cells and with various extracellular matrix components.

“PBMNC” means a peripheral blood-derived mononuclear cell. “PBS” means Phosphate-Buffered Saline, a buffer.

“PLA” means processed lipid aspirates of an adipose (fat tissue). According to implementations of the present disclosure, PLA contains adipose tissue stem/stromal cells (i.e., Ad-MSCs).

“Percentage of cells expressing CD34+, CXCR4+ or CD38−” means an assay of the number of eukaryotic cells expressing CD34+, CXCR4+ or CD38− is quantifiable through measurement of CD34+, CXCR+ or CD38− expression (e.g., via mRNA, FACS or MACS analysis).

“Pericyte” means a mesenchymal-like cell, associated with the walls of small blood vessels. As relatively undifferentiated cells, pericytes serve to support these vessels, but it can differentiate into a fibroblast, smooth muscle cell, or macrophage as well if required.

“Proteome” means the complete set of expressed proteins present in a eukaryotic cell at a given time, assayable via mRNA, MACS and FACS analysis.

“Purified Reishi” means a reishi extract prepared as described in U.S. Publication No. 2007/0104729 A1, incorporated by reference herein, wherein the purified reishi is comprised of a polysaccharide or glycopeptide containing terminal fucose residues.

“Reishi” means the mushroom Ganoderma lucidum, or its close relative Ganoderma tsugae.

“Stem cell” means an unspecialized progenitor cell that gives rise to a specific specialized functioning tissue cell, such as a blood, bone, and nerve cell.

“Stromal Cells” means connective tissue cells of an organ found in the loose connective tissue. These are the cells which make up the support structure of biological tissues and support the parenchymal cells. These are most often associated with the uterine mucosa (endometrium), prostate, bone marrow precursor cells, and the ovary as well as the hematopoietic system and elsewhere.

“Subject” means, but is not limited to, any eukaryotic cell or eukaryotic cell-based organism, whether administered in vivo or in vitro to that cell or cell-based organism.

“Suspension Cells” means incubated eukaryotic cells that have not adhered to the sides of the incubation vessel.

“0.1 N Tris buffer” means a buffer solution.

“VCAM” means a cell surface molecule (otherwise known as CD-106). VCAM-1 promotes the adhesion of lymphocytes, monocytes, eosinophils, and basophils.

Ganoderma lucidum and their extract products have been widely used for many years for health promotion. Previous studies on F3 have shown its role in hematopoietic myeloid and lymphoid activities and antitumor function. It has been proposed that enhancement of tissue cell CAM expression may limit tumor cell metastasis. The inventors of the instant disclosure discovered that F3 exhibits TPO- and GM-CSF-like function to enhance the expressions of CAMs in human tissue stem/progenitor cells.

In one implementation, a method is disclosed comprising homogenizing reishi tissue; dissolving the reishi extract in water; stirring the reishi extract/water mixture for at least about 24 hours while maintaining the temperature of the reishi extract/water mixture at a temperature of at least about 4° Celsius; centrifuging the reishi extract/water mixture for a sufficient amount of time to remove insoluble materials; evaporating water from the centrifuged reishi extract/water mixture at a temperature of at least about 35° Celsius in order to remove at least a portion of the water from the reishi extract/water mixture; lyophilizing the resultant concentrated reishi extract/water mixture; resuspending the lyophilized reishi extract in a liquid phase; and purifying the resuspended reishi extract.

According to implementations, the lyophilized reishi extract is resuspended in 0.1 N Tris buffer. In another implementation, the lyophilized reishi extract is resuspended and the resultant liquid suspension is adjusted to a pH of 7.0. In another implementation, the resuspended reishi extract is purified by use of gel filtration, chromatography, or other purification techniques. In another implementation, the resuspended reishi extract gel filtration chromatography, or other purification is collected as a series of fractions, each of which is subjected to anthrone analysis or the phenol-sulfuric acid method in order to detect sugar components. In another implementation, the filtered resuspended reishi extract is dialyzed to remove excessive salt. In another implementation, the reishi extract is subjected to anion exchange, eluted with sodium chloride solution and optionally re-fractionated. In another implementation, the filtered resuspended reishi extract is re-lyophilized subsequent to filtration.

According to implementations, a method is disclosed comprising combining a plurality of mononuclear cells with purified reishi extract; and incubating the mononuclear cells and reishi extract for a sufficient amount of time so as to increase the mononuclear cell expression of cell markers.

According to implementations, the combination further includes conditional culture medium. In another implementation, the conditional culture medium is AIM-V. In another implementation, the AIM-V conditional medium is serum-free. In another medium, the AIM-V conditional culture medium is supplemented with fetal bovine serum, an equivalent, or a serum substitute. In another implementation, the AIM-V conditional culture medium is supplemented with IL-4 and GM-CSF.

According to implementations, the cell surface adhesion molecules (CAMs) are such as ICAM, ECAM, VCAM or NCAM.

According to implementations, the cell surface markers are selected from the group consisting of immature dendritic cell markers. In another implementation, the immature dendritic cell markers are selected from the group consisting of CD1a, CD14, CD40, CD80, and CD86.

According to implementations, the cell surface markers are selected from the group consisting of mature dendritic cell markers. In another implementation, the mature dendritic cell markers are selected from the group consisting of CD83.

According to implementations, the cell surface markers are selected from the group consisting of hematopoietic cell markers. In another implementation, the primitive CD34+ hematopoeitic stem cell markers are selected from the group consisting of co-expression of CD34+, CD38−, CD133+, or CXCR4+.

According to implementations, the cell surface markers are selected from the group consisting of B cell markers. In another implementation, the B cell markers are selected from the group consisting of CD19 and CD20.

According to implementations, the purified reishi extract (alone or in combination with mononuclear cells) is administrable to a person in need of treatment.

According to implementations, a method is disclosed comprised of administering a sufficient amount of purified reishi extract subject to increase expression of cellular proteins selected from the group consisting of BMP-2, aggrecan, and IL-11.

According to implementations, the combination further includes conditional culture medium. In another implementation, the conditional culture medium is AIM-V. In another implementation, the AIM-V conditional medium is serum-free. In another medium, the AIM-V conditional culture medium is supplemented with fetal bovine serum. In another implementation, the AIM-V conditional culture medium is supplemented with IL-4 and GM-CSF.

According to implementations, purified reishi extract is provided as part of a kit option TGF, chondroitin, or glucosamine skeleton forming agents. In another implementation, a combination is disclosed comprising co-administering the combination reagents with one or more skeleton forming agents with or without a medical device within which the reagents or skeleton forming agents are impregnated to a subject.

According to implementations, the combination of the MSC or PLA cells and purified reishi extract is further comprised of compounds selected from the group consisting of insulin, TGF-B1, or ascorbate-2-phosphate.

According to implementations, a method is disclosed comprising the steps of combining a plurality of MSC or PLA cells in vitro with purified reishi extract; incubating the MSC or PLA cells and reishi extract for an amount of time; and quantitatively assessing the percentage of co-cultured MSC cells that retain CD34+, CXCR4+, or CD38− cell proteome characteristics wherein the percentage of cells expressing CD34+, CXCR4+, or CD38− as a percentage of the total number of cells incubated with reishi extract is increased as compared to the percentage of cells expressing CD34+, CXCR4+, or CD38− as a percentage of an equivalent total number of cells incubated without purified reishi extract.

### F3 Influences Cell Adhesion Molecule (CAM) Expressions in Hematopoietic Mononuclear Cells (MNCs):

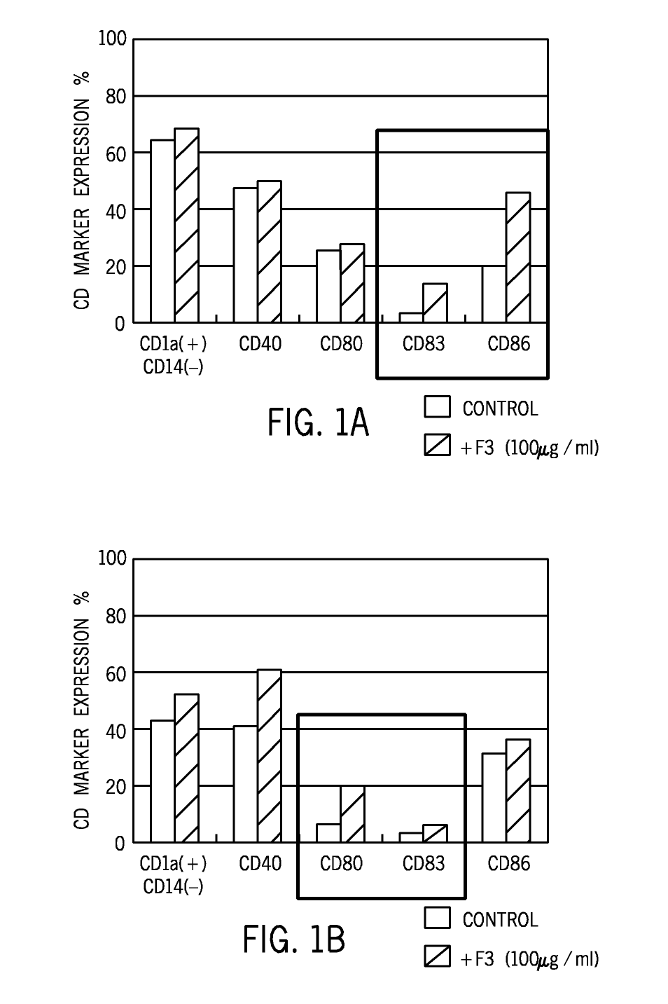

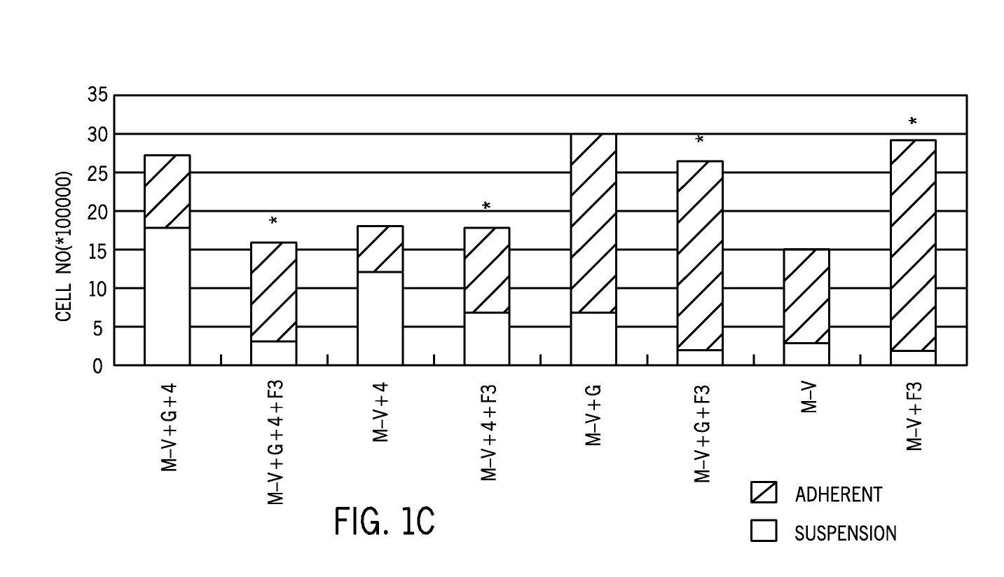

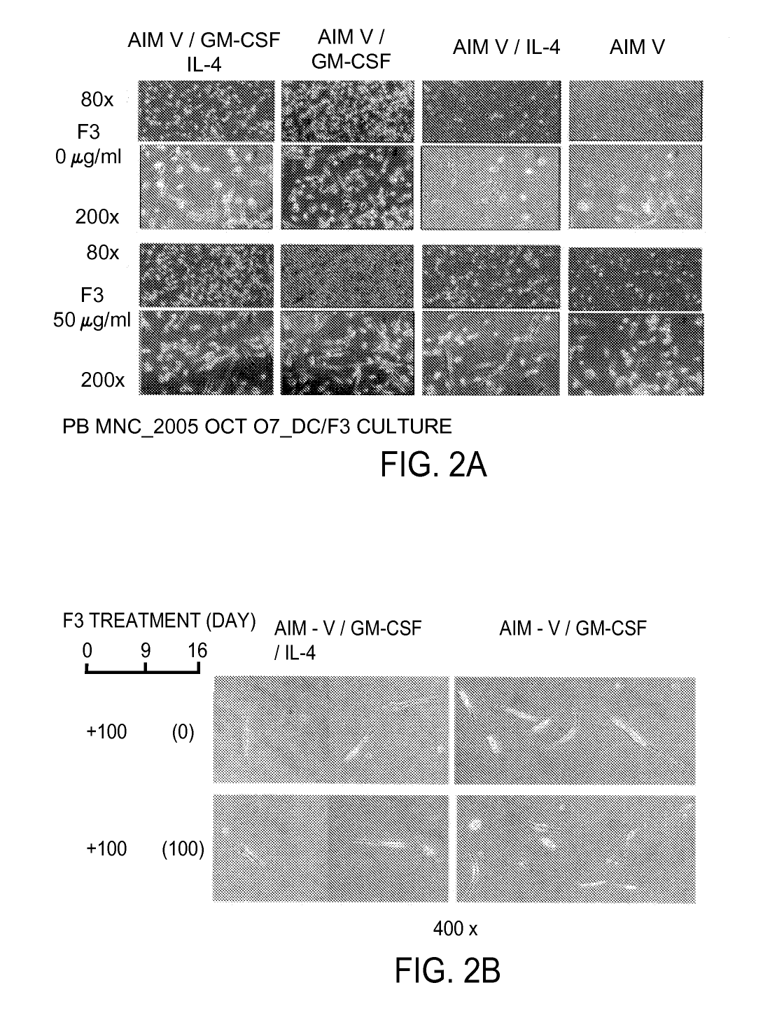

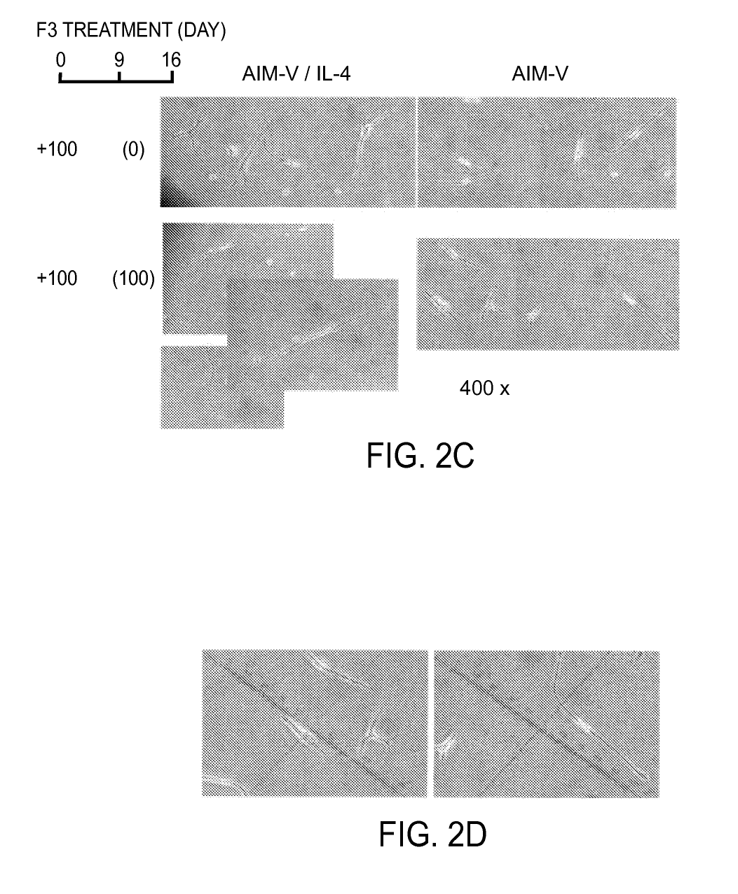

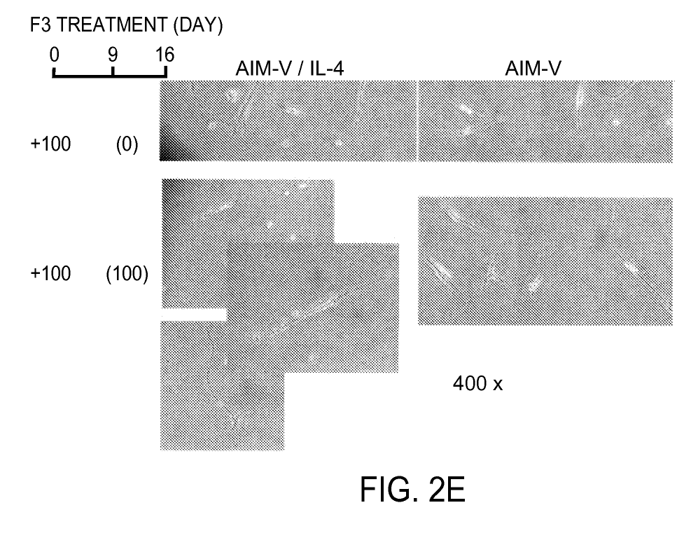

Cell adhesion molecules and co-stimulatory factors of adherent mononuclear cells are known to be important in the activation of eukaryotic immune system cells and anti-cancer metastasis. F3 significantly increased the CB-MNC attachment in the culture dish, and induced cellular morphological changes, regardless of the presence of cytokines (GM-CSF (G), IL-4 (4)) under AIM-V (M-V) basal culture media (FIGS. 1A-1C, 2A-2E).

In FIGS. 1A-1C, F3 increased the adherent DC (relatively immature) subpopulation in AIM-V medium culture, under the presence of IL-4 and GM-CSF. The FACS data shown in F3 increased the relatively immature (DC80+) and mature (CD86+) DCs phenotype CD83(+), CD1a(+), CD40(+), and CD14(−).

According to implementations, FIGS. 2A-2D qualitatively depict the amount of adherence of mononuclear cells markers in vitro when incubated with and without purified reishi, as characterized by visual observation of the relative confluence of the cells on the incubation vessel. According to implementations, FIG. 2E qualitatively depicts the morphological changes in mononuclear cells in vitro when incubated with and without purified reishi, as characterized by visual observation of the morphology of the incubated cells.

### F3 Enhances Immature Dendritic Cells (DCs) Formation and B Cell Production:

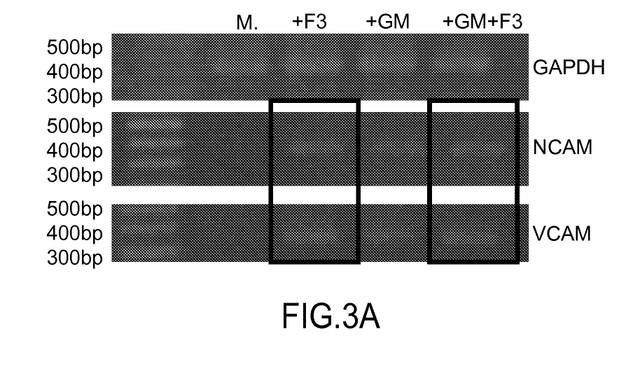

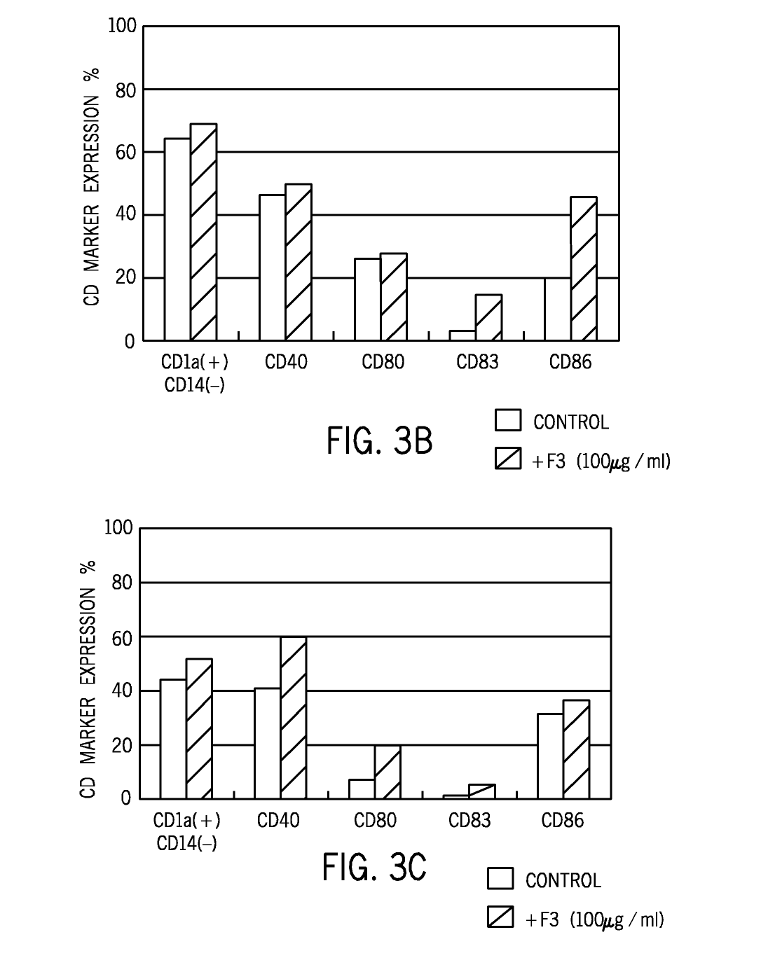

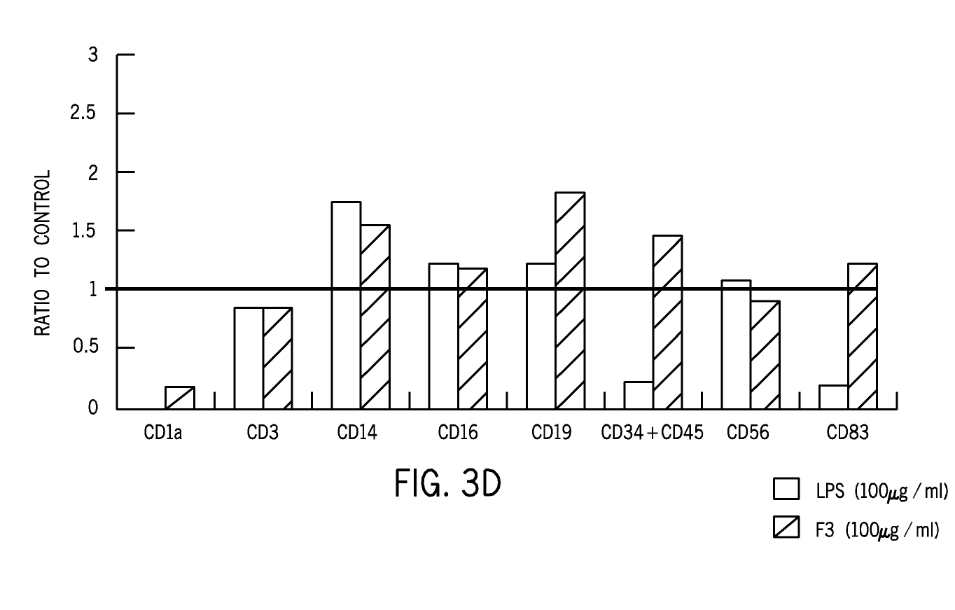

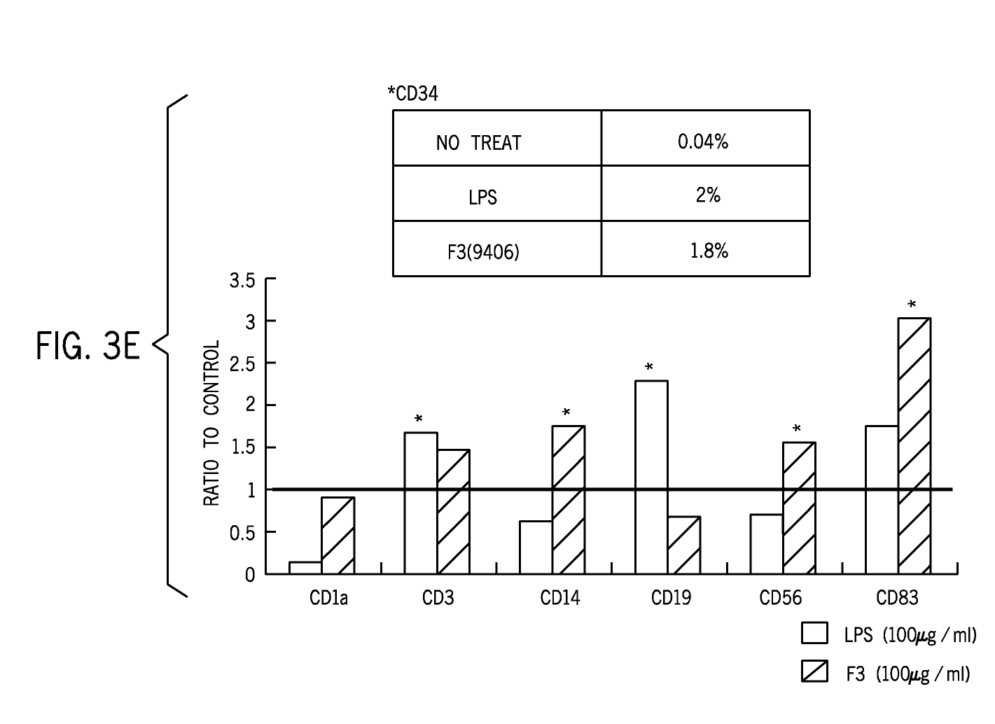

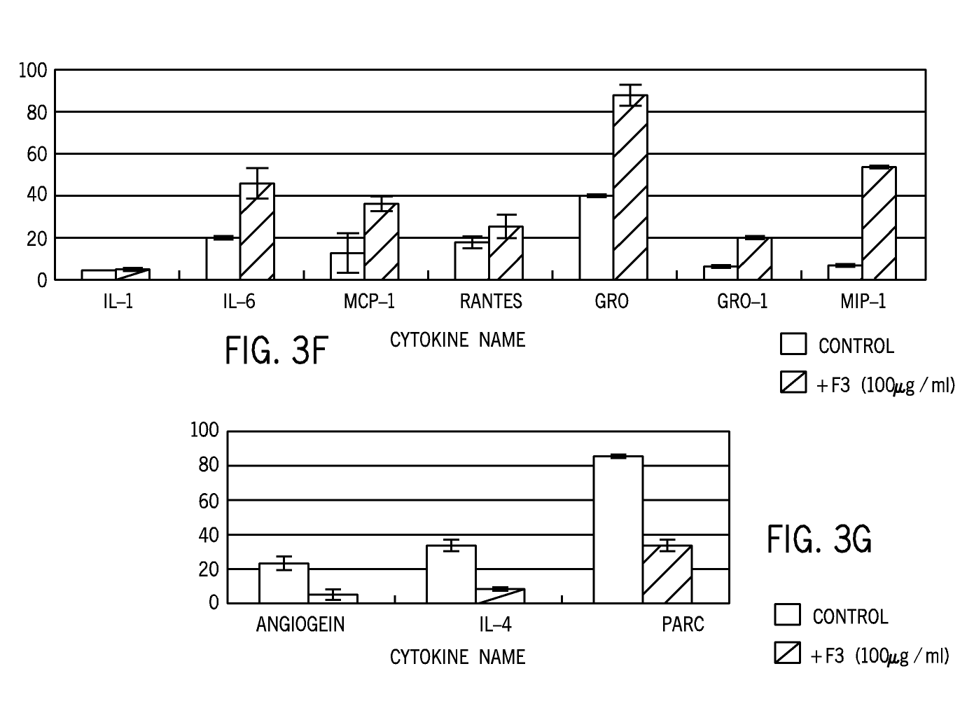

Dendritic cells, the mononuclear cells that initiate immune response, and Granulocyte-macrophage colony-stimulating factor (GM-CSF) and macrophage colony-stimulating factor (M-CSF) together with IL-4 are known to reciprocally regulate the MNC to DC trans-differentiation. Upon examining the influence of F3 on the CB-MNC to DC transformation, F3 (N-CAM) expressions (FIG. 3A), but not the I-CAM nor PE-CAM. Adhesive CB-MNCs stimulated by F3 not only resulted in making more CD83+ mature dendrite cell (DC) in RPMI basal medium (FIG. 3D-E), but also may produce more active immature adherent DC(CD1a+, CD40+, CD80+, and CD86+) subset, with enhanced the cell dendrite growing under the AIM-V medium culture condition (FIGS. 3B-3C). CB- and PB-MNCs respond differently to the F3 stimulation in transforming into immature DC cells.

F3 may serve as an effective adjuvant for transforming monocytes to generate more immature and active DCs for cancer immunotherapy. Furthermore, upon the stimulation of reishi F3, it was also found that the CD19+ subpopulation B cells were significantly increased in CB-MNCs to DC culture (FIG. 3D).

Immature DC-OC trans-differentiation has been shown greatly enhanced by rheumatoid arthritis synovial fluid and involves proinflammatory cytokines such as IL-1 or TNF-α, as well as components of the ECM such as hyaluronic acid (immature dendritic cell transdifferentiation into osteoclasts: a novel pathway sustained by the rheumatoid arthritis microenvironment. (Blood 104:4029-37 (2004), hereby expressly incorporated by reference.) F3 influences the up regulations of pro-inflammatory cytokines and down regulations of the PARK and angiogenin expressions of adult blood, in a dendritic cell culture, under co-stimulations of GM-CSF and IL-4 for 7 days. IL-1β, IL-6, MCP-1, RANTES, GRO, GRO-α, MIP-β were up regulated, as contrasted to the down regulated PARC and angiogenin cytokines analyzed by a human cytokine array (FIGS. 3F-3G).

### F3 Maintenance of the Primitive Hematopoietic CD34+ Stem Cell Population in Vitro Expansion of CB-HSCs:

Sovalat H, et al., J. Hematother Stem Cell Res. 12, 473-489 (2003)(herein expressly incorporated by reference) demonstrated that decreasing adhesion of hematopoietic stem/progenitor cells (HSCs) to BM stromal cells in mobilized blood was due to a down regulation of adhesion molecules of the CD34+ cells and CD34+CD38− proteome subset, in compared to those from steady-state blood, bone marrow, and cord blood.

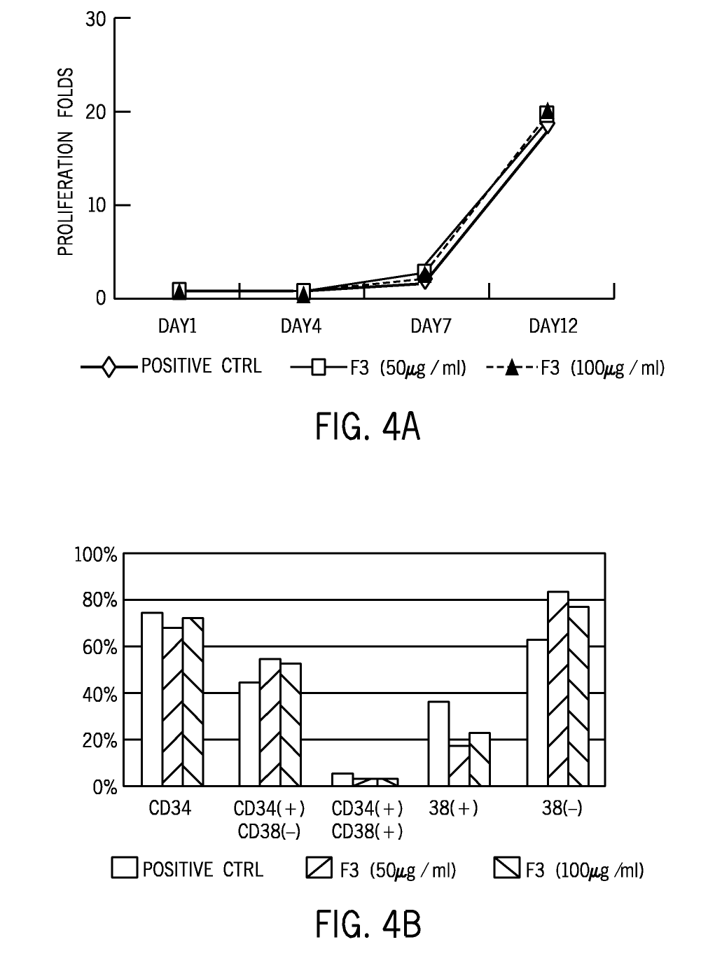

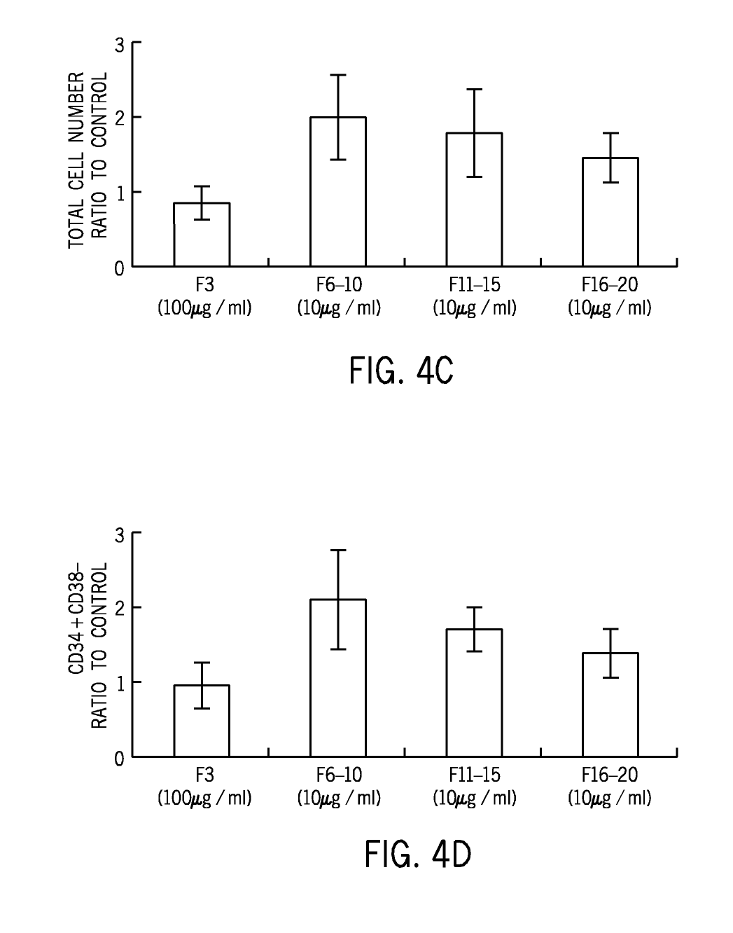

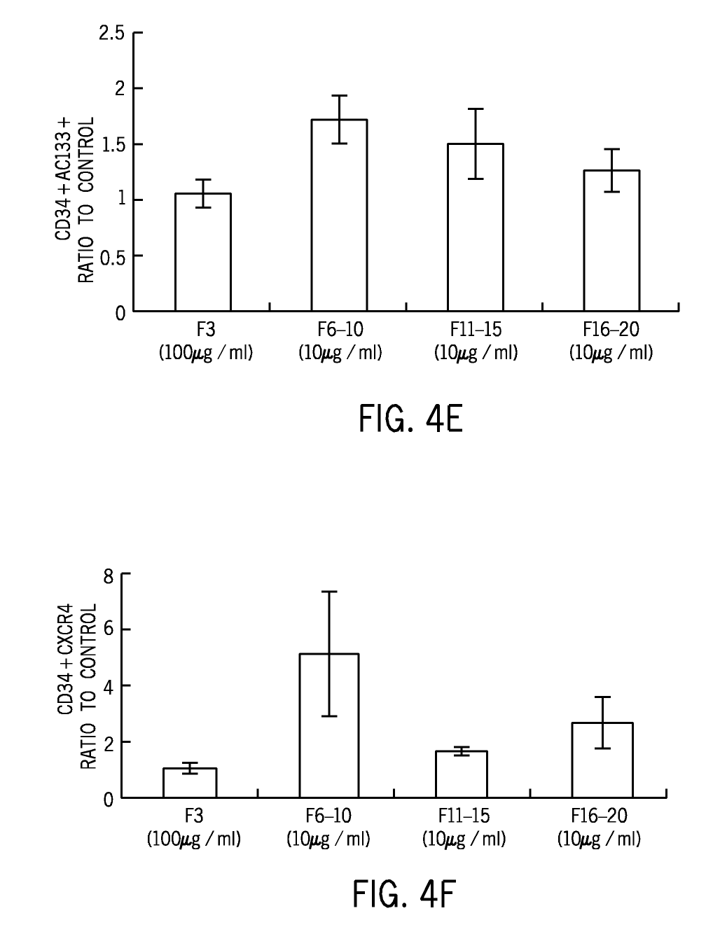

In this study, CD34+ HSCs from CB were isolated and subjected to a five cytokine cocktail liquid culture and a stromal cell based co-culture system to examine the F3 influences of CD34+ HSCs survival and proliferation. As shown in FIGS. 4A-4C, reishi extract and particularly the F3 fraction of reishi extract retains CD38− and CD133+ primitive HSC subpopulations of CD34+ HS/PCs in both liquid and feeder co-culture systems, indicating that F3 may serve as a soluble matrix for preventing the primitive CD38− and CD133+ subpopulations of CD34+ HSCs from differentiation in the ex vivo expansion culture.

By examining the F3 hydrolyzed components, reishi extract fractions have been identified that enhance the CD38− and CD133+ primitive (FIGS. 4D-4E) and CXCR4+ HSC CD34+ (FIG. 4F) populations. These data indicate that the glycoside moiety loss in liquid culture may be prevented by supplementing selective natural glycosides or glycoproteins, such as F3, as soluble scalford for maintaining primitive HSCs in undifferentiated state for the ex vivo expansion culture. Therefore selective glycosides or glycoproteins, such as F3, may be supplemented in ex vivo expansion co-culture of CB-HSCs and treating the leukaphresis treated mobilized PB-HSCs for better transplant success.

### F3 Influences the MSC Chondrogenic Differentiation Potentials

N-CAM has been shown critical in CNS development, involved in neurite out-growth, axon guidance, and migration, and activate signal receptor induce intracellular signal cascades. Fang, J. H. B. Int J. Dev. Bio. 43:335-342 (1999) (hereby expressly incorporated by reference). N-CAM expression has also been shown involved in skeletal condensation and initiating chondrogenesis (mediating the formation of precartilagious condensation) in the early chondrogenesis (Widelitz et. al., J. Cell. Physiol. 156: 399-411 (1993)(hereby expressly incorporated by reference).

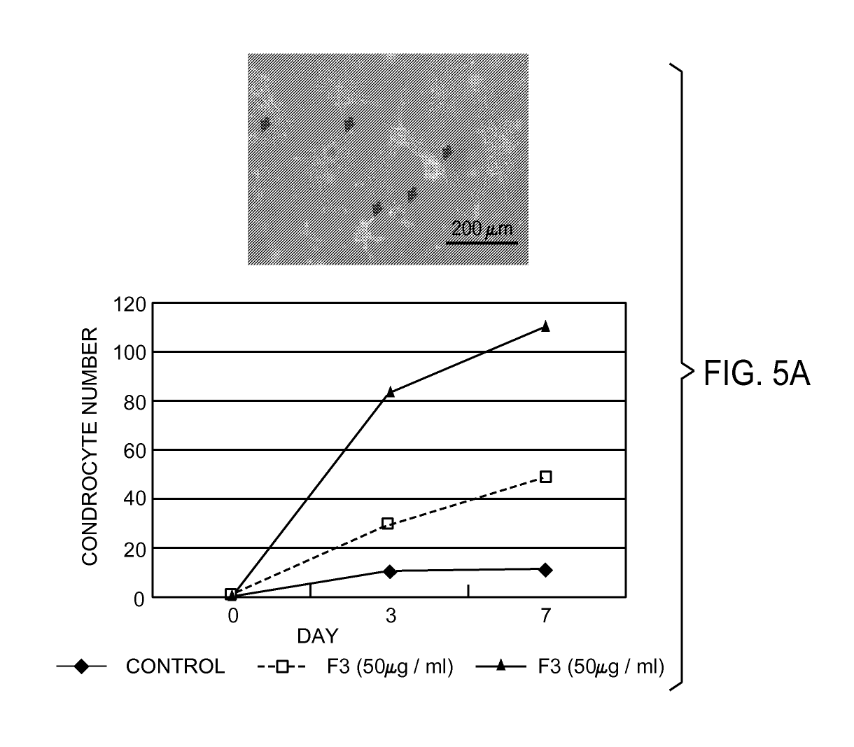

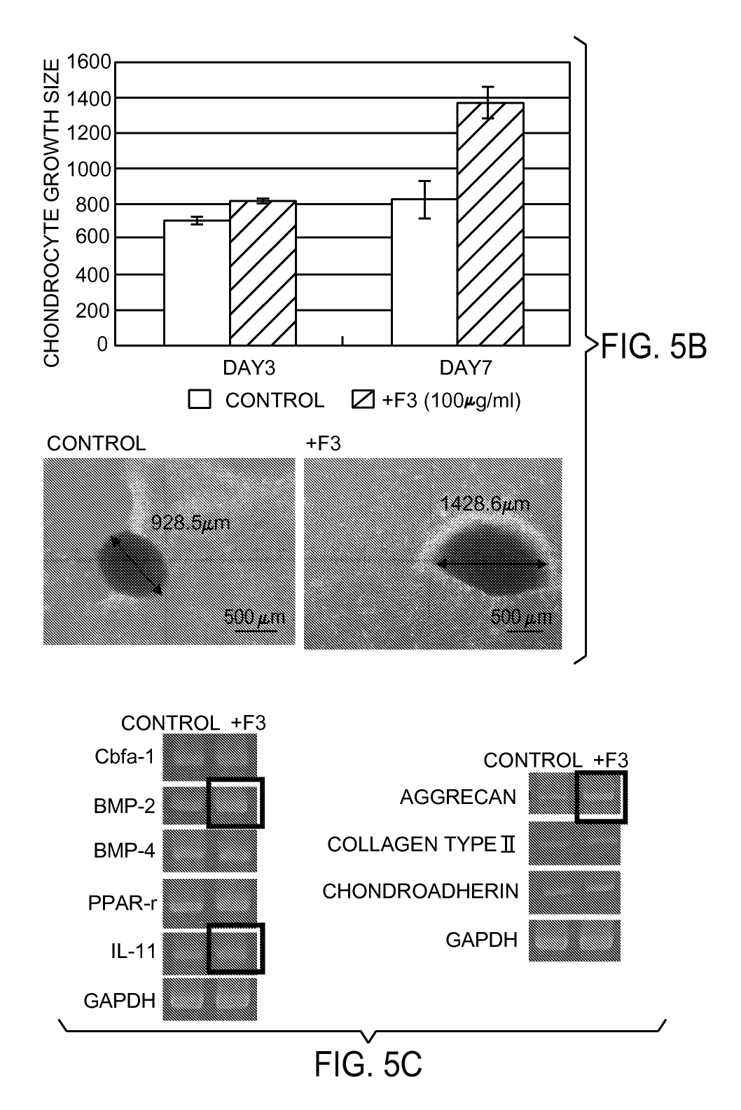

Upon examination of F3 influence in a chondrosphere induction mesenchymal stem cell differentiation culture, it was found that F3 accelerates and enhances mesenchymal chondrosphere formation, accompanied by increased BMP-2, IL-11 and aggrecan gene expressions (FIGS. 5A-5C). Based on these results, F3 may be useful for skeleton remodeling drug by co-formulation with skeleton forming agents.

According to implementations, the polysaccharide fraction of Ganoderma lucidum (F3) was found to enhance human tissue stem/progenitor cell adhesion/homing and the survival/renew. It exhibits TPO and IL-6 cytokine-like function for maintenance & enrichment of the primitive HSC populations, in the ex vivo expansion culture, by enhancing cells survival in quiescent and homing to diminish HSCs from differentiation. F3 exhibits inhibitory effects on blood endothelial progenitor colony (EPC) formation with concomitant reduction of vascular endothelial growth factor-3 (VEGFR-3) and endoglin (CD105) cell markers, under the presence of angiogenic factors. Molecular profiling assay further evidenced that F3 indeed inhibited the angiogenin synthesis and enhanced chemokines, e.g., IL-1, MCP-1, MIP-1, RANTES, and GRO productions, in the EPC derivation experiment. Therefore, F3 may regulate the tissue stem/progenitor cell activities, in many ways: 1) to enhance human tissue stem/progenitor cells adhesions and the survival/renew abilities for stem/progenitor cell culture; 2) to diminish the differentiation and to enhance the survival/renew of primitive hematopoietic stem cells (HSCs) that may promote the engraftment of blood and marrow HSCs in transplantation therapy; and 3) to prohibit tumor metastasis by inhibiting blood leukocyte/progenitors from angiogenesis and lymphangiogenesis.

### F3 Sustains the Primitive HSCs Survival in Quiescent and Undifferentiated State in Expansion Culture.

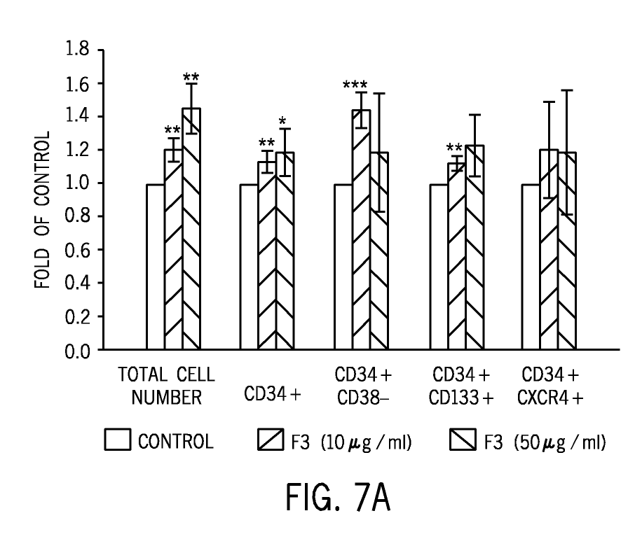

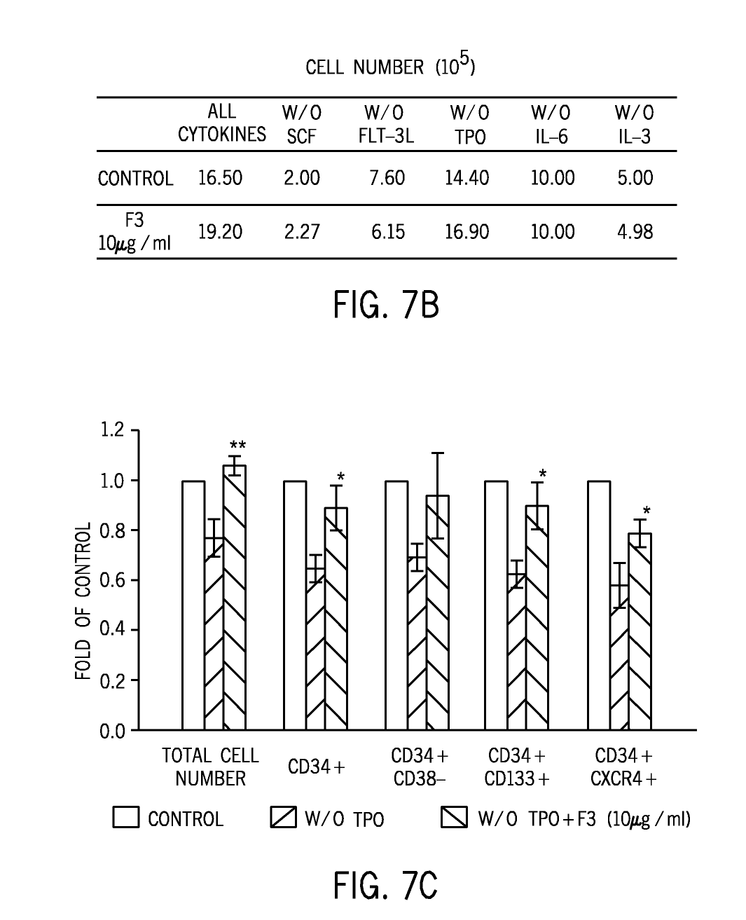

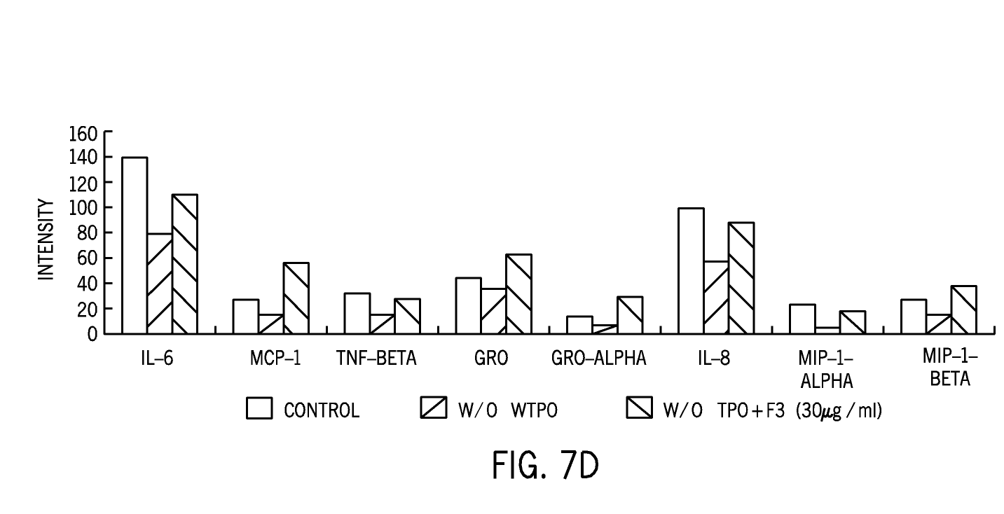

Despite numerous studies on the effect of cytokines on HSCs in vitro, the exact signals that govern cell renewal of undifferentiated HSCs are still not fully understood. However, several cell renewal cytokines proposed for expanding undifferentiated HSCs, including SCF, Flt-3, IL-6, IL-3, and TPO have been used. Multiple lines of evidence indicate that thrombopoietin (TPO) contributes to the transplantation of HSCs by supporting their cell survival and proliferation in vitro and enhances the engraftment of transplanted cells into marrow in vivo. Accordingly, the influence of F3 on human umbilical cord blood hematopoietic stem/progenitor cells regarding their survival and proliferation in a five-cytokine cocktail liquid medium has been examined. The result showed that in the presence of F3, the primitive CD38− and CD133+ HSC subpopulation of CD34+ HSCs in the culture was kept from differentiation and thus useful for the purpose of ex vivo expansion (FIG. 7A). In a BrdU assay, it was shown that the reduction of S phase population with increase in the G0/G1 population is consistent with the increased quiescent primitive HSC cell marker expressions (data not shown). Previous studies showed that TPO is associated with the increase in surviving before entering the G1 phase of CD34+ HSCs to maintain their survival in the undifferentiated state. In a further cytokine replacement culture study, F3 exhibited the TPO like function as shown in FIGS. 7B-7D.

FIG. 7A illustrates the effect of the F3 on HSC expansion culture, according to implementations. F3 maintained the primitive HSCs in a cytokine cocktail culture, as shown by the influence of F3(10 μg/ml) (F3+) on CD34+ HSCs (by fold) in an ex vivo expansion culture (FIG. 7B-FIG. 7C). F3 exhibited TPO like function in the HSC expansion culture, as shown in a cytokine replacement experiment. The cocktail for the ex vivo HSC expansion system comprised SCF, Flt-3L, TPO, IL-6, and IL-3 cytokines in IMDM medium, as shown in FIG. 7B. In FIG. 7D, F3 was found equivalent to TPO and maintained CD38−, AC133+, and CXCR4+ primitive HSC subpopulations, in the expansion culture. (*, P<0.05; **, P<001; ***, P<0.001).

### F3 Effect on Human Monocytic Dendritic Cell (DC) Derivation.

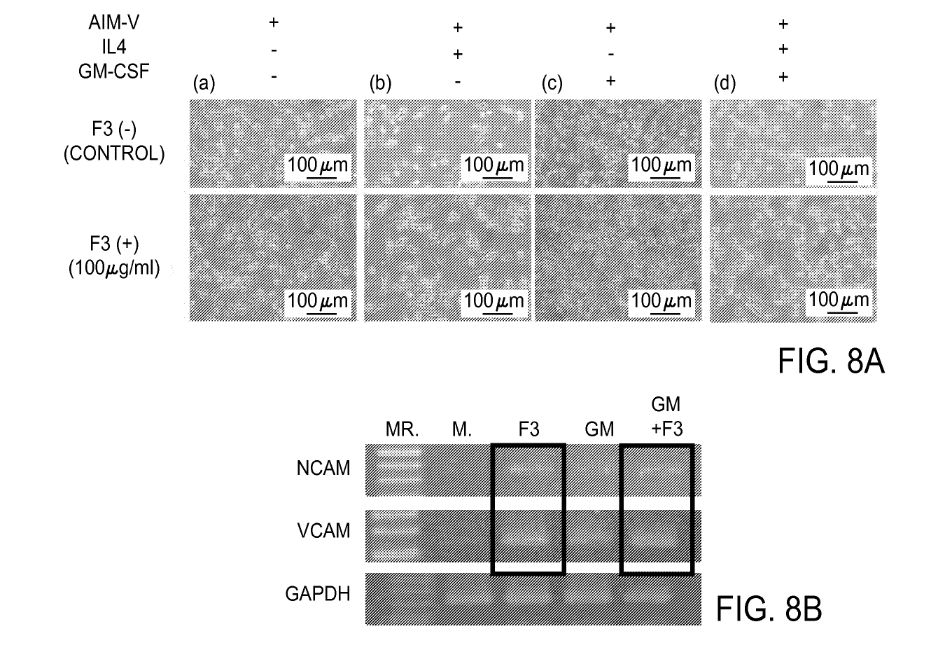

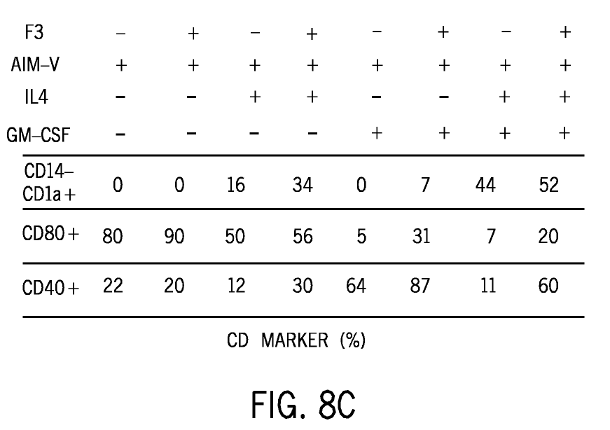

The effectiveness of DC derivation in cancer immune therapy heavily relies on the ex vivo transformation of blood MNCs into active DCs in culture. Adjuvants and co-stimulatory factors are the important sources of MNCs for the presentation of tumor antigen(s) and activation of the immune system. Using two-cell culture systems, the effect of F3 was tested on progenitor monocytic DC transformation. The results showed that F3 significantly increased the adhesive property of blood mononuclear cells (MNCs) in the culture dish, and induced changes in cell morphology, regardless the presence of IL-4 and GM-CSF in the RPMI basal and AIM-V culture media, as shown in the results presented in FIGS. 8A-8C.

Examination of the phenotype of the cultured cells by FACS, it was shown that the blood MNCs stimulated by F3 induced different maturity of DC populations, i.e., more CD83+ mature DCs in the RPMI basal medium, and more adherent immature DCs (CD1a+, CD40+, CD80+, and CD86+) with enhanced cell dendrite growing (FIGS. 8A-8C), in the AIM-V medium. Furthermore, examination of the influence of F3 on the CAM expression of the adherent MNCs, by means of RT-PCR analysis, showed that F3 preferentially enhanced the expression of Ig super family CAMs (e.g., N-CAM, V-CAM) (FIG. 8B), but not PE-CAM or ICAM. The increased cell adhesion and morphologic changes of the cultured cells were consistent with the increased expression of immature DC markers as determined by FACS analysis (FIG. 8C). These data indicate that, using the AIM-V culture condition, F3 serves as an effective adjuvant to produce the relatively immature DC subset with better antigen presentation function, suggesting its potential use in cancer immune therapy.

FIGS. 8A-8C depicts implementations of experimental data showing how F3 affects blood dendritic cell (DC) formation in culture. In FIG. 8A, F3 significantly increased the attachment of MNCs and induced morphology changes in the culture, regardless of the presence of GM-CSF or IL-4 in AIM-V culture media. The transcriptional gene expression of N-CAM and V-CAM is shown in FIG. 8B. The immature surface markers (CD1a, CD40, CD80) was increased by F3 treatment in AIM-V medium, supplemented with GM-CSF (GM) alone or IL-4 (+4) alone or GM-CSF and IL-4 together (+G+4), as shown in FIG. 8C.

### F3 Effect on Human Blood MNC Cytokine Expression

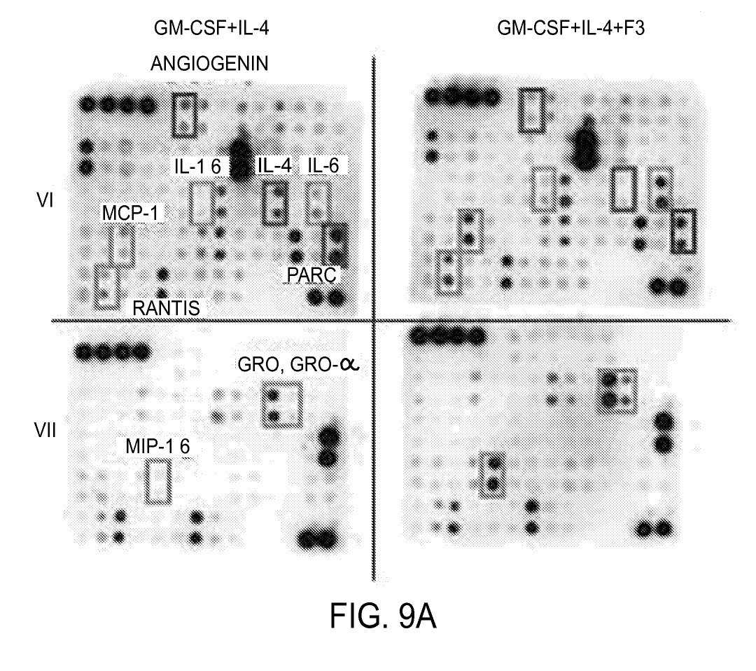

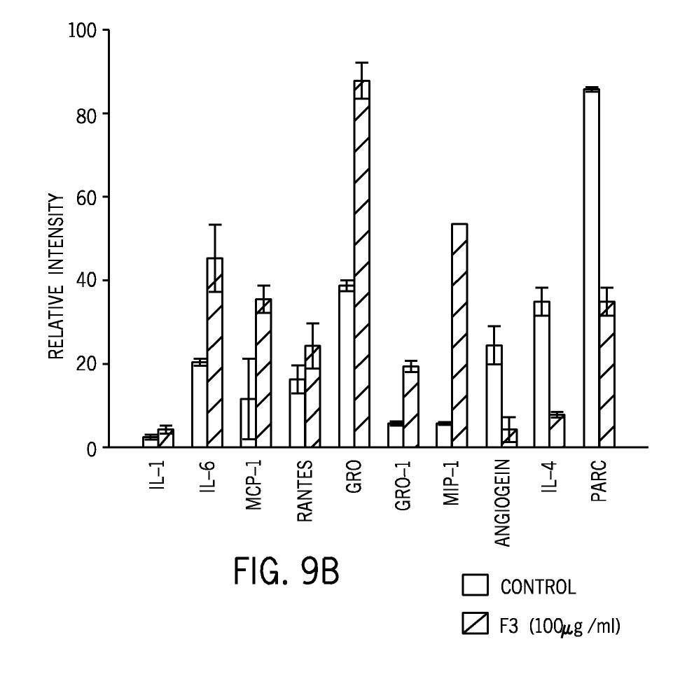

According to implementations, FIGS. 9A-9B depict the F3 effect on human blood MNC cytokine expression. F3 caused a significant decrease in the secretion of angiogenin and PARC and an increase in the levels of IL-6, GRO, MIP-1, MCP-1, and RANTES in blood MNCs.

### F3 Effect on Human Monocytic Endothelial Angiogenesis and Lymph Angiogenesis.

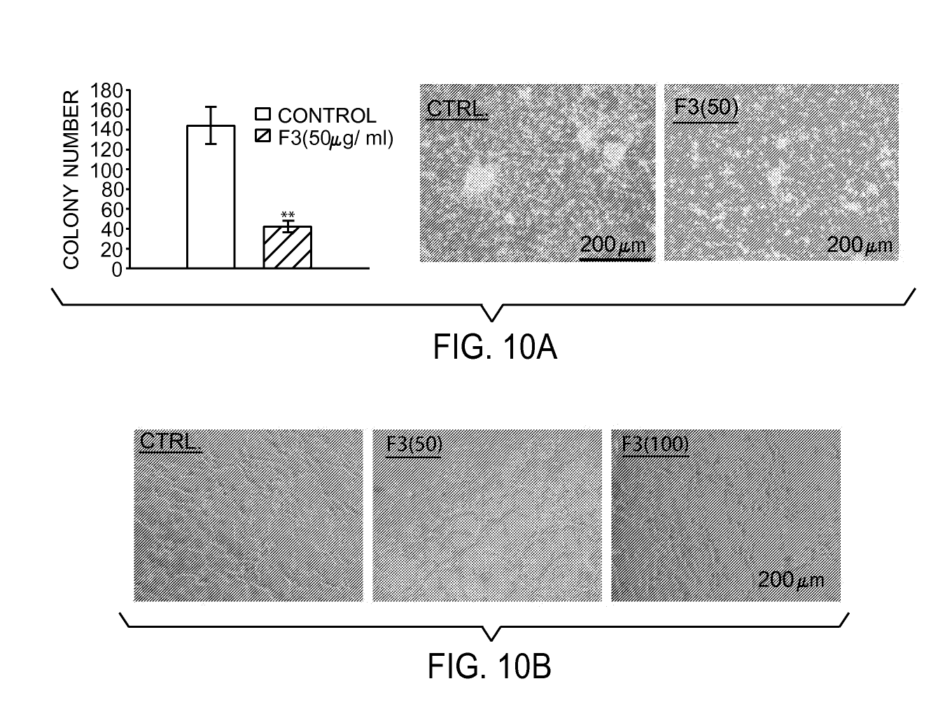

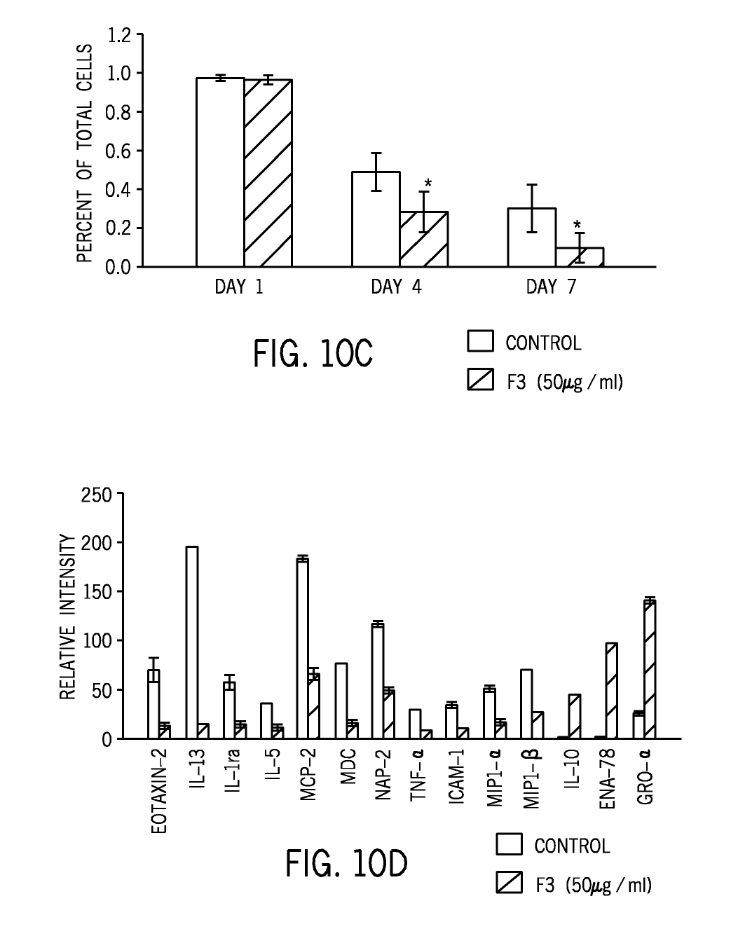

Normal wound healing vasculogenesis is regulated by well-balanced angiogenic and angiostatic factors. Both EPC colony formation assay in liquid culture and matrix gel endothelium cell (EC) tube formation analysis have been used to probe the influence of F3 on angiogenesis. As shown in FIG. 10C, F3 exhibited little influence on EC tube formation. However, it significantly diminished the potential of circulatory EPC colony formation in adult peripheral blood MNCs (PB-MNCs), as shown in FIGS. 10A-10B. In the presence of F3, the cultured cells showed a decrease in the expression of both angiogenic Flt-4 and endoglin (CD105).

A Q-PCR array analysis of the cultured cells verified the down regulation of angiogenic factors (e.g.FLt-1, KDR, VEGFR-3, CD105 and PECAM (CD31)) as shown in Table 2. Both VEGFR-3 (Flt-4) and CD105, are involved in the initiation of neo-vascular angiogenesis and lymph-angiogenesis. The inhibition of EPC colony formation by F3 is likely associated with the angiogenic initiation, instead of subsequent vasculogenesis associated with tube lining and stabilizations.

TABLE 2
Regulation of angiogenic factors caused by F3

| | | Fold of |
|---|---|---|
| Symbol | Protein | Regulation |
| ANGPT1 | Angiopoietin 1 | −15.85 |
| ANGPTL4 | Angiopoietin-like 4 | −6.22 |
| CXCL10 | Chemokine (C—X—C motif) ligand 10 | −261.55 |
| CXCL9 | Chemokine (C—X—C motif) ligand 9 | −365.58 |
| EFNA1 | Ephrin-A1 | −5.76 |
| EFNA3 | Ephrin-A3 | −7.00 |
| EPHB4 | EPH receptor B4 | −7.89 |
| FGF1 | Fibroblast growth factor 1 (acidic) | −16.00 |
| FGFR3 | Fibroblast growth factor receptor 3 | −14.68 |
| | (achondroplasia, thanatophoric dwarfism) | |
| FLT1 | Fms-related tyrosine kinase 1 (vascular | −18.50 |
| | endothelial growth factor/vascular permeability | |
| | factor receptor) | |
| HAND2 | Heart and neural crest derivatives expressed 2 | −5.75 |
| IFNA1 | Interferon, alpha 1 | −27.55 |
| IFNB1 | Interferon, beta 1, fibroblast | −324.02 |
| IL6 | Interleukin 6 (interferon, beta 2) | −6.40 |
| KDR | Kinase insert domain receptor (a type III | −9.13 |
| | receptor tyrosine kinase) | |
| LEP | Leptin | −39.75 |
| MDK | Midkine (neurite growth-promoting factor 2) | −21.99 |
| PLG | Plasminogen | −6.01 |
| PLXDC1 | Plexin domain containing 1 | −11.59 |
| TEK | TEK tyrosine kinase, endothelial (venous | −12.93 |
| | malformations, multiple cutaneous and | |
| | mucosal) | |
| TIMP3 | TIMP metallopeptidase inhibitor 3 (Sorsby | −9.02 |
| | fundus dystrophy, pseudoinflammatory) | |
| TNF | Tumor necrosis factor (TNF superfamily, | −19.62 |
| | member 2) | |
| CXCL1 | Chemokine (C—X—C motif) ligand 1 | 6.82 |
| | (melanoma growth stimulating activity, alpha) | |
| CXCL5 | Chemokine (C—X—C motif) ligand 5 | 59.43 |
| EDG1 | Endothelial differentiation, sphingolipid G- | 5.21 |
| | protein-coupled receptor, 1 | |
| HIF1A | Hypoxia-inducible factor 1, alpha subunit | 7.76 |
| | (basic helix-loop-helix transcription factor) | |
| PECAM1 | Platelet/endothelial cell adhesion molecule | 155.90 |
| | (CD31 antigen) | |
| PROK2 | Prokineticin 2 | 5.26 |
| TGFA | Transforming growth factor, alpha | 5.09 |
| THBS1 | Thrombospondin 1 | 85.70 |

According to implementations of experimental data, FIGS. 10A-10D depict the F3 effect on angiogenesis. In FIGS. 10A-10B, F3 reduces the blood EPCs colony formation in culture (FIG. 10A) but does not affect the AdMSCs derived EC tube forming activities (FIG. 10B). In FIG. 10C, F3 inhibits, dose dependently, the expression of VEGFR3 and endoglin (CD105) as analyzed by FACS. In FIG. 10D F3 was shown to up regulate IL-10, ENA-8, and GRO and down regulate Eotaxin-2, IL-13, IL-1ra, MCP-2, MCD, MIP-2, TNF-a, and ICAM-1 secretions.

### F3 Influences the Condrogenic Differentiation Potential of Adipose Mesenchymal Stem/Stromal Cells (Ad-MSCs)

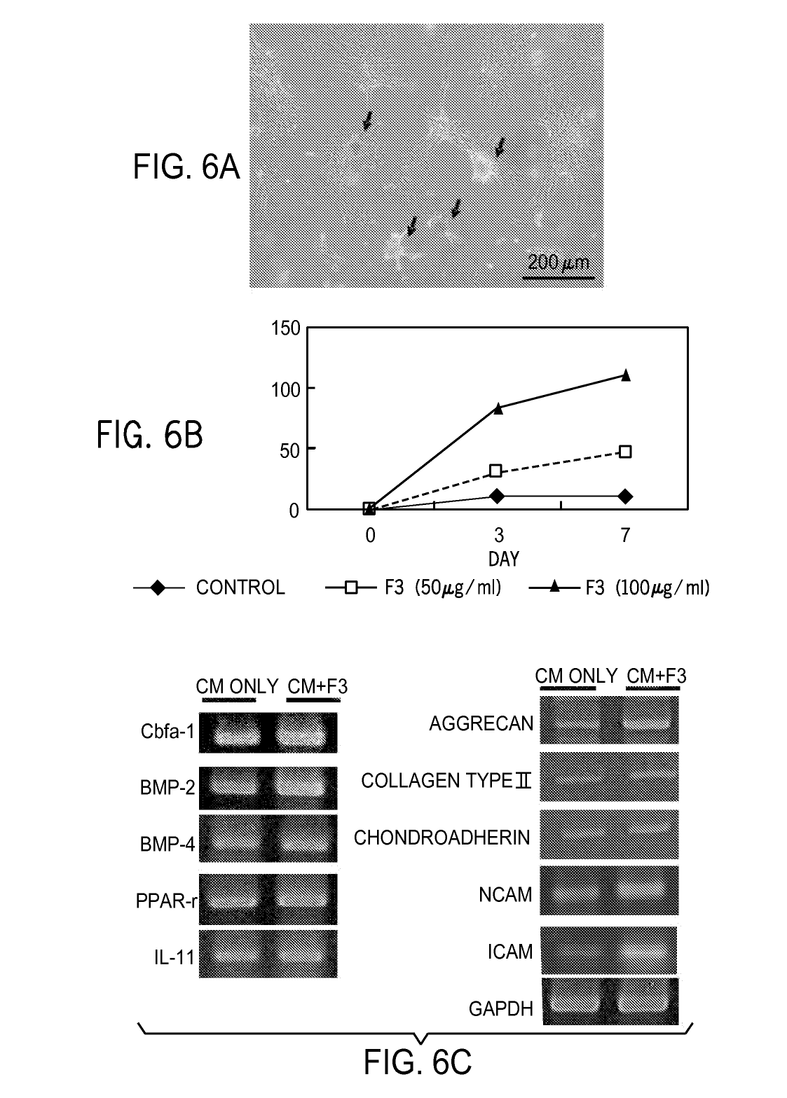

N-CAM was first found in central nervous system development, involved in neurite out-growth, axon guidance, and migration, and activates receptor-mediated intracellular signal cascades. N-CAM expression is also involved in precartilaginous skeletal condensation and initiation of condrosphere formation in the early stage of chondrogenesis. When Ad-MSCs were subjected to condrogenesis in the presence of F3, it was found that F3 accelerates and enhances the mesenchymal chondrosphere formation, and is accompanied with increase in the expression of N-CAM, ICAM, IL-11(VLA-4), BMP-2, and aggrecan (FIGS. 6A-6C and FIG. 5C). This result implies that F3 is likely specifically targeting the skeletal condensation initiation of MSCs, because in an osteogenic formation assay, no significant contribution of F3 to the osteoblast formation and the late phase bone formation was observed, as analyzed by von Kossa staining and calcium incorporation assay.

### Enhancement of Survival and Engrafting Potential of HSCs in Culture.

In the presence of G-CSF, marrow mobilized PB-HSCs were shown to have a significant decrease in transplant efficiency, due to their loss of homing receptor (CXCR4). On the other hand, the low engrafting rate of umbilical cord blood (CB) transplantation was associated with its low CD56+ natural killer (NK) cells, as compared to PB-HSC. Therefore, to obtain a better engraftment from mobilized or cultured HSCs, transplanting cells with primitive stem cell phenotype (such as CD34+, AC133+, Lin-, and CD38−) and homing receptors (such as CXCR4 and CD44) are desired. TPO has been proposed for therapeutic use, but due to its immune mediated complication, it is currently not used therapeutically. F3 exhibits TPO like function so that the primitive CD38− and CXCR4+ subset of CD34+ HSCs can be retained in the HSCs expansion culture. The data obtained are consistent with the proposal that stromal cell N-CAM functions as a HSC homing associated molecule.

### Angiogenic Inhibitory Effect and Lymphangiogenic Prevention.

The reduction of EPC colony formation by F3 was elucidated by the cytokine array and Q-PCR array data, showing the decreased expression of monocytic angiogenin, Flt-1, KDR, endoglin (CD105) and VEGFR-3 (Flt-4) (Table 2). Because angiogenin expression was found specifically in the early pregnant normal uterine decidual cells, not in the late gestation vasculogenesis, it was predicted that the inhibitory effect of F3 on EPC initiation and angiogenic factors inhibit tumor growth and metastasis.

### Enhancement of the Condrosphere Formation of Connective Tissue MSCs.

The effect of F3 on connective tissue stem/progenitor cells accelerates and enhances the early condrosphere formation of Ad-MSCs, with a concomitant increase in expression of BMP-2, IL-11, N-CAM, I-CAM, and aggrecan genes. These results indicate that F3 is likely targeting the mesenchymal progenitor cell aggregation at the initial stage of the condrogenesis, by enhancing the expression of autokines (e.g., aggrecan, BMP-2, and IL-11) and CAMs. F3 has been shown to have no significant contribution to the osteoblast formation and the late phase bone formation, as analyzed by von kossa staining and calcium incorporation assay. Therefore, it may be advantageous to combine F3 with other compounds to benefit the precartilaginous condensation for the joint repair and skeleton remodeling.

Thus, F3 influences tissue stem/progenitor cells by i) maintaining the CD38− CD34+ primitive hematopoietic stem cell subset in culture, ii) sustaining the immature dendritic cell formation to improve antigen presentation, iii) inhibiting EPC colony formation, and iv) promoting MSC aggregation and condrosphere formation.

### Pharmaceutical or Nutraceutical Compositions

According to another aspect, the reishi or fractions thereof can be included in a pharmaceutical or nutraceutical composition together with additional active agents, carriers, vehicles, excipients, or auxiliary agents identifiable by a person skilled in the art upon reading of the present disclosure.

The pharmaceutical or nutraceutical compositions preferably comprise at least one pharmaceutically acceptable carrier. In such pharmaceutical compositions, the Ganoderma lucidum or fractions thereof form the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite, chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Subject as used herein used in conjunction with a pharmaceutical or nutraceutical composition or method refers to humans and non-human primates (e.g., guerilla, macaque, marmoset), livestock animals (e.g., sheep, cow, horse, donkey, and pig), companion animals (e.g., dog, cat), laboratory test animals (e.g., mouse, rabbit, rat, guinea pig, hamster), captive wild animals (e.g., fox, deer), and any other organisms who can benefit from the agents of the present disclosure. There is no limitation on the type of animal that could benefit from the presently described agents. A subject regardless of whether it is a human or non-human organism may be referred to as a patient, individual, animal, host, or recipient.

Pharmaceutical compositions suitable for an injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

According to implementations, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected location to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of the active compound (i.e., an effective dosage) may range from about 0.001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

### EXAMPLES

### Example 1

### Materials

Crude reishi extract (prepared via alkaline extraction (0.1 N NaOH), neutralization and ethanol precipitation) was obtained from Pharmanex Co. (CA, USA). Immobiline DryStrip (pH3-10 NL (non-linear), 18 cm) and IPG buffer (pH3-10 NL) were purchased from Amersham Pharmacia Biotech (Uppsala, Sweden). CHAPS, Tris buffer, agarose, iodoacetamide and alpha-cyano-4-hydroxycinnamic acid were from Sigma Co. (St. Louis, Mo., USA); dithioerythritol (DTE) was from Merck Co. (Darmstast, Germany); acrylamide, ammonium persulfate (APS) and TEMED were from Bio-Rad (Hercules, Calif., USA); sodium dodecyl sulfate (SDS) and glycine were from Fluka (Buchs, Switzerland); sequencing grade trypsin was from Promega (Madison, Wis., USA).

Crude reishi extract (prepared via alkaline extraction (0.1 N NaOH), neutralization and ethanol precipitation) was obtained from Pharmanex Co. (CA, USA). All the chemicals and reagents were from Sigma Co. (81. Louis, Mo., USA) unless indicated.

### Example 2

### Purification of Reishi Extract

According to implementations, twenty eight mg of the crude extract were dissolved in 2 mL of Tris buffer (pH7.0, 0.1 N) and centrifuged to remove the insoluble materials (7 mg). The supernatant was purified by gel filtration chromatography using a Sephacryl S-500 column (100 cm×1.6 cm) with 0.1 N Tris buffer (pH7.0) as the eluent. The flow rate was set at 0.5 mL/min, and 7.5 mL per tube was collected. After the chromatography, each fraction was subjected to anthrone analysis to detect sugar components. Five fractions were collected (fractions 1-5), each dialyzed to remove excessive salt and lyophilized to give 1.0, 6.2, 5.3, 2.1, and less than 1 mg, respectively.

According to implementations, one hundred grams of crude reishi extract were dissolved in 3 L of double distilled water, stirred at 4° C. for 24 h, and centrifuged for 1 h to remove the insoluble. The resulting solution was concentrated at 35° C. to give a small volume and lyophilized to generate 70 g powder of dark-brown color, 2.5 g of which were dissolved in a small volume of Tris buffer (pH7.0, 0.1 N) and purified by gel filtration chromatography using a Sephacryl S-500 column (95 cm×2.6 cm) with 0.1 N Tris buffer (pH7.0) as the eluent. The flow rate was set at 0.6 mL/min, and 7.5 mL per tube was collected. After the chromatography, each fraction was subjected to anthrone analysis or the phenol-sulfuric acid method to detect sugar components.

Five fractions were collected (fractions 1-5), each dialyzed to remove excessive salt and lyophilized to give 450 mg of fraction 3.

Fraction 3 was further subjected to a column of Diaion-W A30 anion exchanger (Cl-form, 40 cm×3.5 cm) eluted with 0.2 and 0.8 M NaCl at a flow rate of 0.5 mL/min and two fractions were designated as F3G1 (11% yield based on fraction 3) and F3G2 (10% yield based on fraction 3), respectively. Another fraction (F3G3, 11% yield based on fraction 3) was generated when the column was further eluted with 2 M NaOH.

The gel-filtration chromatography of F3G2 was carried out on a TSK HW-75 column (130 cm×2.6 cm) eluted with double distilled water at a flow rate of 0.5 mL/min. There were two fractions collected; i.e., G2H1 (19% yield based on F3G2) and G2H2 (69% yield based on F3G2).

According to implementations, the crude reishi extract (100 g) was dissolved in 3 liters of double distilled water, stirred at 4° C. for 24 h, and centrifuged for 1 h to remove the insoluble components. The resulting solution was concentrated at 35° C. to give a small volume and lyophilized to generate 70 g of dark-brown powder, 2.5 g of which were dissolved in a small volume of Tris buffer (pH7.0, 0.1 N), and purified by gel filtration chromatography using a Sephacryl S-500 column (95×2.6 cm) with 0.1 N Tris buffer (pH7.0) as the eluent. The flow rate was set at 0.6 ml/min, and 7.5 ml per tube was collected. After the chromatography, each fraction was subjected to phenol-sulfuric acid test to detect sugar components. Five fractions were collected (fractions 1-5) and combined, dialyzed to remove excessive salt and lyophilized to give 450 mg of F3.

### Example 3

### Anthrone Colorimetric Method

Each 1.5 mL of anthrone (9,10-dihydro-9-oxoanthracene) solution (0.2 g anthrone dissolved in 100 mL of conc. sulfuric acid) in a series of test tubes immersed in an ice water bath was carefully overlayed with 1.5 mL of sample (20-40 μg/mL of D-glucose or equivalent). After all additions had been made, the tubes were shaken rapidly and then replaced in an ice water bath. The tubes were heated for 5 min in a boiling water bath and then cooled; the optical densities were read within an hour at 625 nm against distilled water. Standards, reagent blanks, and unknowns were run in triplicate because of likely contamination by other carbohydrate sources. Calculations were made on the basis that the optical densities are directly proportional to the carbohydrate concentration.

### Example 4

### Mitogen-Induced Proliferation of Spleen Cells and Colorimetric MTT Assay

Whole spleen cells were harvested from BALB/c male mice (six weeks old), suspended in RPMI-1640 medium containing 10% FCS (fetal calf serum), and centrifuged to remove the supernatant. The collected precipitated cells were first suspended in 1 mL of RBC lysis buffer (8% NH₄Cl), then 14 mL more of the same lysis buffer were added to destroy red blood cells. After 1 min, the solution was diluted with 15 mL RPMI-1640 medium to stop the reaction, centrifuged to collect the cells, and adjusted the cell final concentration to 2×10⁶ cells/mL with RPMI-1640 medium. Concanavalin A (Con A, final conc: 1 μg/mL) was added to the resulting mixture. The cells were incubated with or without a reishi extract (or partially purified fraction) in 96-well ELISA plates at 37° C. with 5% CO₂ for 72 h. The cell proliferation was measured based on the MTT assay.

MTT (3-(4,5-dimethylthiazol-2-yl)-2,5-diphenyl tetrazolium bromide) was dissolved in phosphate buffered saline (PBS) at 5 mg/mL and filtered to sterilize and remove a small amount of insoluble residue present in some batches of MTT. At the times indicated below, 25 μL of MTT solution was added to all wells of an assay, and plates were incubated at 37° C. for 4 h. Acid-isopropanol (100 μL of 0.04 N HCl in isopropanol) was added to all wells and mixed thoroughly to dissolve the dark blue crystals. After a few minutes at room temperature to ensure that all crystals were dissolved, the plates were read on a Perkin Elmer ELISA reader (HTS 7000 plus), using a test wavelength of 570 nm, a reference wavelength of 620 nm. Plates were normally read within 1 h after the addition of isopropanol.

### Example 5

### Reverse Transcription (RT) and Polymerase Chain Reaction (PCR)

Mouse spleen cells were aseptically removed from healthy mice (BALB/c male mice, six weeks old), adjusted to an ideal cell concentration (4×10⁶ cells/mL) and incubated in RPMI-1640 medium containing 10% of FCS (fetal calf serum) at 37° C. with 5% CO₂. After 6 h, the cells were subjected to RNA extraction using Qiagen RNAeasy mini kit to obtain ˜1 μg of the desired RNA. Reverse transcription (RT) was performed using the Thermoscript R/T PCR System, and the Themoscript system protocol I, from Gibco BRL. The reaction was carried out as follows: 8 μL of RNA, 2 μL of primer (Oligo(dT)₂₀), 2 μL of 10 mM dNTP Mix, and DEPC H₂O (0.1% diethylpyrocarbonate-treated H₂O) was added to each tube, which was then incubated at 65° C. for 5 min and immediately put on ice. The following was added to each tube as a 8 μL mixture: 4 μL of 5× cDNA buffer, 1 μL of 0.1 M dithiothreitol (DTT), 1 μL of RNaseOut (a ribonuclease inhibitor), 1 μL of Thermoscript R/T, and 1 μL of DEPC water. The mixture was incubated at room temperature for 10 min and then 55° C. for 30 min to allow first strand of cDNA synthesis. Enzyme activity was terminated by incubating the reactions at 85° C. for 5 min and the tubes were then placed on ice for 10 min. The samples were stored at −20° C. until used for PCR.

Each sample (3 μL) was added to each reaction tube and the following reagents were added as a 47 μL mix: 5 μL of 10×PCR buffer, 4 μL of 10 mM dNTP Mix, 2 μL of each primer (10 OD/mL, sense and anti-sense), 33 μL of DEPC H₂O, and 1 μL of ProZyme® (DNA polymerase, from PROtech Technology). The reaction tubes were placed in a Strategene PCR Robocycler (Gradient 96) and run under the following condition: 1 cycle at 92° C. for 2 min (initial denaturation), then 30 consecutive cycles of 91° C. for 10 s (denaturation), 59° C. for 25 s (primer annealing), and 72° C. for 25 s (primer extension). The reactions were analyzed by gel electrophoresis.

### Example 6

### Reverse Transcription (RT) and Polymerase Chain Reaction (PCR)

Total cellular RNA was isolated using RNeasy® Total RNA isolation kit (Qiagen) and cDNA was synthesized using the SuperScript™ First-strand Synthesis System (Life Technologies). Specific genes were amplified by PCR reaction using Fast-Run Taq Master Kit (Protech Technology). The primer sequences used for gene amplification were shown in the list below.

According to implementations, mouse spleen cells were aseptically removed from healthy mice (BALB/c male mice, six weeks old), adjusted to an ideal cell concentration (3×10⁶ cells/mL) and incubated in RPMI-1640 medium containing 10% of FCS (fetal calf serum) at 37° C. with 5% CO2. After 6 h, the cells were subjected to RNA extraction using Qiagen RNAeasy mini kit to obtain ˜1 μg of the desired RNA. Reverse transcription (RT) was performed using the Thermoscript R/T PCR System, and the Thermoscript system protocol I, from Gibco BRL. The reaction was carried out as follows: 1 μg of RNA, 1 μL of primer (Oligo(dT)20) and 2 μL of 10 mM dNTP Mix were added to each 0.2 mL tube and adjusted the total volume to 12 μL with DEPC H₂O (0.1% diethylpyrocarbonate-treated H₂O). The mixture was incubated at 65° C. for 5 min and immediately chilled on ice. The following was added to each tube as an 8 μL mixture: 4 μL of 5× cDNA buffer, 1 μL of 0.1 M dithiothreitol (DTT), 1 μL of RNaseOut (a ribonuclease inhibitor), and 1 μL of Thermoscript R/T, and 1 μL of DEPC water. The mixture was incubated at room temperature for 10 min and then 50° C. for 1 h to allow first strand of cDNA synthesis. Enzyme activity was terminated by incubating the reactions at 85° C. for 5 min and the tubes were then placed on ice for 10 min. The samples were stored at −20° C. until used for PCR.

Each sample (2 μL) was added to each reaction tube and the following reagents were added as a 25 μL mix: 2.5 μL of 10×PCR buffer, 2 μL of 10 mM dNTP mix, 2.5 μL of 10 mM each primer (sense and anti-sense), 13 μL of DEPC H₂O, and 1 μL of ProZyme® (DNA polymerase, from PROtech Technology). The reaction tubes were placed in a Strategene PCR Robocycler (Gradient 96) and run under the following condition: 1 cycle at 94° C. for 2 min (initial denaturation), then 25 consecutive cycles of 94° C. for 1 min (denaturation), primer annealing (various temperatures depending on primers, see Tables 3 and 4 for details) for 1 min and 72° C. for 1 min (primer extension). The reactions were analyzed by gel electrophoresis.

TABLE 3
Primer Sequences Used in RT-PCR Experiments

| | | | Annealing | Size of PCR |
|---|---|---|---|---|
| | | Seq. ID | temp | product |
| Cytokine | Sequences (S: sense, A: anti-sense) | No | (° C.) | (bp) |
| IL-1β | S 5′-CAACCAACAAGTGATATTCTCCATG-3′ | 1 | 55 | 152 |
| | A 5′-GATCCACACTCTCCAGCTGCA-3′ | 2 | | |
| IL-2 | S 5′-TGATGGACCTACAGGAGCTCCTGAG-3′ | 3 | 59 | 167 |
| | A-5′GAGTCAAATCCAGAACATGCCGCAG-3′ | 4 | | |
| IL-4 | S 5′-ACGAGGTCACAGGAGAAGGGACGCCATGCA-3′ | 5 | 71 | 221 |
| | A 5′-TCTTCATGGAGCAGCTTATCGATGAATCC-3′ | 6 | | |
| IL-6 | S 5′GTGACAACCACGGCCTTCCCTACT-3′ | 7 | 53 | 313 |
| | A 5′-GGTAGCTATGGTACTCCA-3′ | 8 | | |
| IL-12 | S 5′-TGTTGTAGAGGTGGACTGG-3′ | 9 | 67-65 | 483 |
| | A 5′-TGGCAGGACACTGAATACTT-3′ | 10 | | |
| IFNγ | S 5′-TGGAGGAACTGGCAAAAGGATGGT-3′ | 11 | 63 | 336 |
| | A 5′-TTGGGACAATCTCTTCCCCAC-3′ | 12 | | |
| TNF-α | S 5′-GCGACGTGGAACTGGCAGAAG-3′ | 13 | 65 | 383 |

TABLE 4
Primer Sequences Used in RT-PCR Experiments

| | | Annealing | Product |
|---|---|---|---|
| gene name | Sequence | temperature | size (bp) |
| Aggrecan | (s)GCCTTGAGCAGTTCACCTTC | 57° | 372 |
| | (SEQ. ID NO: 14) | | |
| | (a)CTCTTCTACGGGGACAGCAG | | |
| | (SEQ. ID NO: 15) | | |
| BMP-2 | (s)ACTTTTGGACACCAGGTTGG | 60° | 379 |
| | (SEQ. ID NO. 16) | | |
| | (a)AGCCACAATCCAGTCATTCC | | |
| | (SEQ. ID NO. 17) | | |
| BMP-4 | (s)GAATGCTGATGGTCGTTTTATTA | 60° | 217 |
| | (SEQ. ID NO. 18) | | |
| | (a)GACGGCACTCTTGCTAGGCT | | |
| | (SEQ. ID NO: 19) | | |
| CBFA-1 | (s)TGCCTGCCTGGGATCTGTAA | 60° | 305 |
| | (SEQ. ID NO. 20) | | |
| | (a)GGACGAGGCAAGAGTTTCAC | | |
| | (SEQ. ID NO: 21) | | |
| Chondroadherin | (s)ACCTGGACCACAACAAGGTC | 57° | 388 |
| | (SEQ. ID NO: 22) | | |
| | (a)CACCTTCTCCAGGTTGGTGT | | |
| | (SEQ. ID NO: 23) | | |
| Collagen type | (s)GAACATCACCTACCACTGCAAG | 57° | 387 |
| II | (SEQ. ID NO: 24) | | |
| | (a)GCAGAGTCCTAGAGTGACTGAG | | |
| | (SEQ. ID NO: 25) | | |
| GAPDH | (s)AATCCCATCACCATCTTCCA | 57° | 318 |
| | (SEQ. ID NO: 26) | | |
| | (a)TGTGGTCATGAGTCCTTCCA | | |
| | (SEQ. ID NO: 27) | | |
| ICAM | (s)ACCCAGTGAGGCCTTATTCC | 60° | 221 |
| | (SEQ. ID NO: 28) | | |
| | (a)TGATCACTGCAGGAAACTGG | | |
| | (SEQ. ID NO: 29) | | |
| IL-11 | (s)ATGAACTGTGTTTGCCGCCTG | 61° | 228 |
| | (SEQ. ID NO: 30) | | |
| | (a)GAGCTGTAGAGCTCCCAGTGC | | |
| | (SEQ. ID NO: 31) | | |
| NCAM | (s)CTCGGCCTIIGTGTTTCCAG | 60° | 341 |
| | (SEQ. ID NO: 32) | | |
| | (a)TGGCAGGAGATGCCAAAGAT | | |
| | (SEQ. ID NO: 33) | | |
| PPARγ | (s)TTCAGAAATGCCTTGCAGTG | 60° | 332 |
| | (SEQ. ID NO: 34) | | |
| | (a)CACCTCTTTGCTCTGCTCCT | | |
| | (SEQ. ID NO: 35) | | |
| VCAM | (s)ATGAGGGGACCACATCTACG | 60° | 232 |
| | (SEQ. ID NO: 36) | | |
| | (a)ATCTCCAGCCTGTCAAATGG | | |
| | (SEQ. ID NO: 37) | | |

According to the RT-PCR studies for the cytokine expression (Table 5), the treatment with F3G2 led to significant expression of all the ten cytokines aforementioned, which was thus concluded to contain the major active components of fraction 3. The expression of TNF-α and IL-1 were detectable in the studies of F3G1 and F3G3. It is of interest that both fractions can trigger only the inflammatory pathway, unlike fraction 3 or F3G2.

TABLE 5
The cytokine expression of mouse splenocytes treated with various reishi
samples, assayed via analysis of total RNA (e.g., RT-pCR).

| | | cytokine expression^{b} | | | | | | | | | |
|---|---|---|---|---|---|---|---|---|---|---|---|
| entry | samples | 1L-1β | IL-2 | IL-4 | IL-6 | INF-γ | IL-12 | TNF-α | GM-CSF | G-CSF | M-CSF |
| 1 | crude reishi | + | − | − | ± | ± | + | + | − | + | + |
| | extract | | | | | | | | | | |
| 2 | fraction 3 | + | − | − | + | + | + | + | + | + | + |
| 3 | F3G1 | + | − | − | − | − | + | + | − | + | ± |
| 4 | F3G2 | + | − | − | + | + | + | + | + | + | + |
| 5 | F3G3 | + | − | − | − | − | ± | + | − | ± | ± |

^{a}Each sample was elevated at four concentrations including 10², 10³, 10⁰ and 10⁴ μg/mL. The cells (3 × 10⁶ cells/mL.) were incubated at 37° C. with 5% CO₂
^{b}=, indicating a significant increase of cytokine expression; −, indicating no increase of cytokine expression; ± showing an increase but not significant of cytokine mRNAs.

The additional gel-filtration chromatography of F3G2 on a TSK HW-75 column resulted in two fractions—G2-H1 (19% yield based on F3G2) and G2H2 (69% yield based on F3G2), as shown in FIGS. 6A-6C. In FIGS. 6A-6C, F3 promotes and accelerates mesenchymal chodrosphere formation, and increases BMP-2, IL-1, and aggrecan gene expressions in Ad-MSC condrogenesis cell culture. The preliminary result from the RT-PCR studies revealed that the former fraction contains much higher activity than the same dosage of F3G2 and G2H2 in the expression of IL-1β, IL-6, INF-γ, TNF-α, and GM-CSF.

### Example 7

### Sugar Composition Analysis—TMS Method

For monosaccharide analysis, the polysaccharide extracts/fractions were methanolyzed with 0.5 M methanolic-HCl (Supelco) at 80° C. for 16 h, re-N-acetylated with 500 μL of methanol, 10 μL of pyridine, and 50 μA of acetic anhydride, and then treated with the Sylon HTP® trimethylsilylating reagent (Supelco) for 20 min at room temperature, dried and redissolved in hexane. GC-MS analysis of the trimethylsilylated derivatives was carried out using a Hewlett-Packard (HP) Gas Chromatograph 6890 connected to a HP 5973 Mass Selective Detector. Samples were dissolved in hexane prior to splitless injection into a HP-5MS fused silica capillary column (30 m×0.25 mm I.D., HP). The column head pressure was maintained at around 8.2 psi to give a constant flow rate of 1 mL/min using helium as carrier gas. Initial oven temperature was held at 60° C. for 1 min, increased to 140° C. at 25° C./min, to 250° C. at 5° C./min, and then increased to 300° C. at 10° C./min.

According to implementations, the carbohydrate composition analyses of crude reishi extract indicated that glucose and mannose exist as the major components together with smaller amounts of other sugars, including fucose, N-acetylglucosamine, xylose, and rhamnose (Table 6).

TABLE 6
Carbohydrate composition of crude reishi extract,
assayed via the TMS method.

| Sugar components | Percentage (%) |
|---|---|
| D-glucose | 58.0 |
| D-mannose | 15.5 |
| L-fucose | 9.7 |
| D-galactose | 9.3 |
| D-xylose | 5.4 |
| D-GlcNAc | 1.0 |
| L-Rham | 0.5 |

The crude extract contains 15.6% proteins, the amino acid analysis of which was shown in Table 7.

TABLE 7
The amino acid composition of crude reishi extract.

| Amino | Relative | | Relative |
|---|---|---|---|
| acid | abundance | Amino acid | abundance |
| Asp | 117 | Met | 6 |
| Thr | 66 | lle | 36 |
| Ser | 54 | Leu | 55 |
| Glu | 120 | Tyr | 16 |
| Pro | 60 | Phe | 28 |
| Gly | 108 | His | 12 |
| Ala | 100 | Lys | 21 |
| Val | 61 | Arg | 22 |

According to implementations, the carbohydrate composition analyses of crude reishi extract, fraction 3, F3G1, F3G2, and F3G3 all indicated that glucose and mannose exist as the major components together with smaller components of other sugars including fucose, galactose, N-acetylglucosamine, and xylose (Table 8). It is of interest that the percentage of galactose decreased significantly in F3G2 and F3G3.

TABLE 8
Relative carbohydrate compositions of various purified reishi fractions,
assayed via the TMS method

| | Percentage (%) | | | | | |
|---|---|---|---|---|---|---|
| | L-Fucose | D-Xylose | D-Mannose | D-Galactose | D-GlcNAc | D-Glucose |
| fraction 3 | 7.1 | 3.1 | 15.1 | 13.5 | 1.20 | 58.1 |
| F3G1 | 8.0 | 5.7 | 10.2 | 12.6 | 0.25 | 63.2 |
| F3G2 | 6.2 | 4.5 | 18.3 | 5.3 | 0.78 | 64.9 |
| F4G3 | 8.4 | 7.2 | 14.5 | 2.9 | 1.18 | 65.7 |

To further understand the composition and activity of fraction 3, it was treated with protease K to partially destroy the protein component. The result showed that proliferation of Con A-stimulated spleen cells remained the same. Glycolytic cleavage by α1,2-fucosidase, however, abolished the activity of fraction 3 completely (based on MTT assay). In contrast, the activity of fraction 3 was slightly reduced after treatment with α1,3/4-fucosidase. This experiment establishes that the active component is a polysaccharide or glycopeptide containing terminal fucose residues with α1,2-fucosidic linkages. Overall, the main active component is a glycoprotein containing essential terminal fucose residues with α1,2-linkages. The protein moiety is not required for the activity.

### Example 8

### Amino Acid Composition Analysis

A sample of crude reishi extract (6 mg) was dissolved in 1 mL solution of 6 M HCl and TFA (4/1), and heated at 140° C. for 3 h. The mixture was concentrated to give a dry residue and dissolved in 100 μL citrate buffer. A small aliquot (4 μL) was withdrawn and subjected to composition analysis by amino acid analyzer (Jeol JLC-6AH).

### Example 9

### Sample Preparation for Proteomic Studies

Reishi extract-treated mouse spleen cells were lysed in 350 μL of lysis buffer containing 8 M urea, 2% CHAPS, 65 mM DTE, 2% v/v isocratic pH gradient (IPG) buffer pH3-10 NL (non-linear), and a trace of bromophenol blue. The sample was centrifuged for 10 min at 13,000 rpm. The total protein concentration in the sample was measured using Bio-Rad protein concentration assay kit. Samples equal to 500 μg of proteins were loaded on immobilized pH gradient strips (pH3-10 NL, 18 cm) for 2-dimensional electrophoresis.

### Example 10

### Two-Dimensional Electrophoresis and Image Processing

The separations were performed as described by Hochstrasser et al. The isoelectric focusing was carried out in an IPGPhor apparatus (Amersham Pharmacia Biotech). The second dimension was done in 10-15% polyacrylamide gradient gels using the Protean II xL 2D multi cell (Bio-Rad). Protein spots were stained with fluorescence dye Sypro Ruby™ (Molecular Probes).

Sypro Ruby-stained gels were scanned with fluorescence laser scanner (Bio-Rad) generating 10 Mb image. The images were analyzed with ImageMaster™ software (Amersham Pharmacia Biotech). The spots in each gel were detected and quantified automatically, using default spot detection parameters. Manual spot editing was performed in agreement with the visual inspection of the gels. The relative volume was calculated in order to correct any differences in protein loading and gel staining.

### Example 11

### MALDI-TOF MS Analysis

Sypro Ruby-stained protein spots were cut from the gel and washed with 200 μL of 50 mM ammonium bicarbonate, pH 8.5, buffer in 50% CH₃CN. Following dehydration in CH₃CN and speed vacuum centrifugation, the gel pieces were swollen in a digestion buffer containing 100 mM ammoniun bicarbonate, pH 8.5, 1 mM CaCl₂, 10% CH₃CN and 50 ng of sequencing grade trypsin. The resulting peptides were extracted with 50% CH₃CN/5% TFA after overnight digestion. A 1 μL aliquot of peptide mixture was deposited on the MALDI target 96-well plate and after few seconds 1 μL of a matrix solution (α-cyano-4-hydroxycinnamic acid in 50% CH₃CN/0.1% TFA) was added. The mixture was allowed to dry at ambient temperature. Positive-ion mass spectrum was measured on a MALDI reflection time-of-flight mass spectrometer MALDI (Micromass UK, Manchester, UK) equipped with a nitrogen laser. The reported spectra were accumulated from 50 to 100 laser shots.

### Example 12

### General Procedure of Fucosidase Treatment

A sample of 10 mg of reishi extract or fraction 3 in 50 mM citrate buffer (pH 6.0) was treated with α1,2- or α1,3/4-fucosidase (5 Unit) at 37 C for a period of time (2-12 h). The mixture was heated in boiling water for 5 min to destroy the enzyme activity, dialyzed against H₂O at 4° C., and lyophilized to give a dry powder for activity studies.

### Example 13

### Mononuclear Cell Isolation

According to implementations, to isolate mononuclear cells (MNCs), each umbilical cord blood (CB) or human peripheral blood (PB) unit was diluted 1:1 with phosphate-buffered saline (PBS)/2 mM EDTA, and carefully loaded onto Ficoll-Hypaque solution. After density gradient centrifugation at 2,000 rpm for 40 minutes at room temperature, MNCs were removed from the interphase and washed two to three times with PBS/EDTA.

According to implementations, MNCs of adult peripheral and umbilical cord blood were obtained from Taipei Blood Donation Center and Taipei Municipal Wan-Fang Medical Center, following the institutional IRB guidelines. MNCs were isolated from the buffy-coat layer by density gradient centrifugation, following the standard procedure. Briefly, each blood unit was diluted 1:1 (v/v) with phosphate-buffered saline in 2 mM EDTA (PBS/EDTA), and carefully loaded onto the Ficoll-Hypaque solution (GE Healthcare). After density gradient centrifugation at 2,000 rpm for 40 minutes at room temperature, MNCs were removed from the interface and washed two to three times with PBS/EDTA.

Monocytic dendritic cell derivation. 2×10⁷ MNCs were plated in 25 cm² flasks in 4 ml RPMI or AIM-V (Gibco) basal medium. After one hour of incubation at 37° C. in a humidified atmosphere containing 5% carbon dioxide, non-adherent cells were removed, and the remaining adherent cells were further cultured in RPMI or AIM-V (Gibco) medium supplemented with human cytokine IL-4 (50 ng/ml) and GM-CSF (50 ng/ml) with/without F3 for 7 days.

### Example 14

### Circulation Endothelial Progenitor Cell (cEPCs) Derivation

The derivation of cEPCs from freshly prepared individual MNCs were preceded by the standard protocol reported previously. Briefly, 2×10⁷ adult PB-MNCs were plated on a 6-well culture plate (pre-coating with 0.1% gelatin) in 1.5 ml of M199 (Clone Tech) medium. After one hour incubation at 37° C. in 5% CO₂ and humidified atmosphere, non-adherent cells were removed. The remaining adherent cells were further cultured in a condition medium of endothelium cells. PB-MNCs derived cEPCs were grown into colony like units, comprising multiple thin, flat cells emerging from a cluster of round cells by day 4 and harvested on the 7^{th} day.

### Example 15

### Dendritic Cell (DC) Culture

2×10⁷ PBMNCs were plated on 25 cm² tissue culture flasks (Cloning) in 4 ml RPMI (Invitrogen) medium after 1 h incubation at 37° C. in humidified atmosphere containing 5% carbon dioxide, nonadherent cells were removed and adherent cells were cultured in serum-free AIM-V medium supplemented with human cytokine IL-4 and GM-CSF (R&D) with/without F3. During culture 7 days period, PBMNCs were transferred to dendritic cell morphology.

### Example 16

### HSC ex vivo Expansion in a Co-Culture System

The CD34+ cells were enriched using a magnetic activated cell sorting (MACS) CD34 isolation kit. Usually 2-5×10⁵ CD34+ cells could be obtained from 1×10⁸ MNCs with 90-95% purity, as confirmed by FACS analysis. Murine stomal cell line (MS-5) was used as a stromal layer for the co-culture of CD34+ cells. MS-5 was cultured more than one week in low-glucose DMEM supplemented with 10% FBS, and PSN antibiotics. The purified CD34+ cells density of 1×10⁴ cells/mL were resuspended in 4 mL serum-free ex vivo 10 medium supplemented with 2 mmol/L 1-glutamine, PSN antibiotics, 10 U/ml recombinant human thrombopoietin (rhTPO; R&D), 50 ng/mL stem cell factor (SCF; R&D), 50 ng/mL flt3-ligand (FL; R&D systems, Minneapolis, Minn., USA), and 10 ng/mL interleukin-6 (Il-6, R&D systems, Minneapolis, Minn., USA). The purified CD34+ cells co-culture with MS-5 feeder in 25 T flask. At day 8, the suspended cells were harvested after the plates had been gently shaken. The remaining weakly attached hematopoietic cells were completely recovered by adding 3 mL DMEM to each well. The total cell and viable cell numbers were estimated using a hematocytometer by counting the number of trypan blue unstained cells under an optical microscope.

### Example 17

### AD-MSCs Isolation and Chondrogenesis

According to implementations, AD-MSCs were obtained from raw human lipoaspirates and cultured as described in Chien et. al., Bioorganic & Medicinal Chemistry 12, 5603-5609 (2004), herein expressly incorporated by reference. Briefly, raw lipoaspirates were washed extensively with sterile phosphate-buffered saline (PBS) to remove contaminating debris and red blood cells. Washed aspirates were treated with 0.075% collagenase (type I; Sigma-Aldrich, St. Louis, Mo.) in PBS for 30 min at 37° C. with gentle agitation. The collagenase was inactivated with an equal volume of DMEM/10% fetal bovine serum (FBS) and the infranatant centrifuged for 10 min at low speed. The cellular pellet was resuspended in DMEM/10% FBS and filtered through a 100 μm mesh filter to remove debris. The filtrate was centrifuged as detailed above and plated onto conventional tissue culture plates in maintain medium. AD-MSCs were maintain in DMEM-LG (GIBCO) supplemented with 10% FBS (Hyclone) AD-MSCs were maintain in DMEM-LG (GIBCO) supplemented with 10% FBS (Hyclone). In vitro differentiations of MSCs were treated with chondrogenic medium: DMEM-LG supplemented with 1% FBS, 6.25 μg/ml insulin, 10 ng/ml TGF-β1 (R&D), and 50 nM ascorbate-2-phosphate. For chondrogenic differentiation, a higher cell density of 1-2×10⁵ per 10 μl was used for chondrosphere formation. During culture chondrocytes were counted at 3 days and at 7 days.

According to implementations, the isolation of Ad-MSCs from freshly prepared individual fat tissues were proceeded by the standard protocol reported previously with some modifications. Briefly, the tissues were washed three to four times with PBS and suspended in an equal volume of PBS (37° C.) (supplemented with 1% bovine serum and 0.1% collagenase type 1 (Sigma)) for 20 min in a shaker incubator. After centrifugation at 400×g for 10 minutes, the pellets were treated with RBC lysis buffer (0.2% Tris base, 0.75% NH₄Cl, pH7.4) for 10 min at room temperature. Suspended cells were passed through a 100 μm cell sieve (Becton Dickinson). The collected cells were seeded at the density of 1×10E5 in each non-coating 10-cm culture plate (Falcon) containing 6-7 ml of DMEM (Gibco) low-glucose basal medium supplemented with 10% FBS (Hyclone) to grow the cells to 75% confluent, before harvesting the Ad-MSCs. All protocols were reviewed and approved by the IRB committee of Taipei Medical University. Liposuction aspirates from subcutaneous adipose tissue site were obtained from donors with a signed consent.

MSC condrogenic differentiation. To initiate the condrogenic differentiation, Ad-MSCs were seeded with a higher cell density of 2×10⁵/10 μl in a DMEM low-glucose medium (Gibco) supplemented with 1% FBS (Hyclone), 6.25 μg/ml insulin (Sigma), 10 ng/ml TGF-β (R&D) and 50 nM ascorbate-2-phosphate (Sigma). After 3 days and 7 days, chondrospheres were observed and examined by microscopy and detected by Alician Blue stain.

### Example 18

### FACS Analysis

According to implementations, adult peripheral blood derived dendritic cells and cord blood expanded HSC were characterized by FACS for specific surface antigens. Harvested cells were collected and stained with fluorescein isothiocyanate- or phycoerythrin-conjugated anti-marker monoclonal antibodies in 100 μL phosphate buffer using titers for 15 minutes at room temperature, as suggested by the manufacturer. Cell surface markers included dendritic cells markers (CD1a, CD40, CD80, CD83, CD86) and hematopoietic lineage markers (CD34, CD38, CD133, CXCR4). Cells were analyzed using a flow cytometry system (FACSCalibur; Becton, Dickinson). Positive cells were counted and compared with the signal of unstained cells.

According to implementations, cells were analyzed according to the instructions of Calibur flow cytometry manufacture (Becton Dickinson). Briefly, an aliquot of 1×10⁵ cells harvested from culture dish were labeled with fluorescent isothiocyanate—(FITC) or phycoerythrin-conjugated (PE) monoclonal antibodies in 100 μl phosphate buffer for 15 minutes at room temperature. Cell surface markers (CD1a, CD40, CD80, CD83, CD86) and hematopoietic lineage markers (CD34, CD38, CD133, CXCR4) were analyzed and quantified.

### Example 19

### Human Cytokine Array Analysis

According to implementations, collected PB-MNCs were transferred into dendritic cells during 7 days conditional medium. The culture medium is AIM-V supplemented with human cytokine IL-4 and GM-CSF (R&D) with or without F3. Cell-secreted cytokines were detected by RayBio™ human cytokine antibody array kit (RayBiotech, Norcross, Ga.). The cytokine antibody array membrane was blocked 30 mins at room temperature. The blocking solution was removed then 2 ml conditional medium incubated with membrane 1.5 hr. The membrane was washed with wash buffer, then biotin-conjugated antibody was added to membrane for 1 hr. After washing, HRP-conjugated streptavidin was added to membrane for 40 mins, then X-ray film exposed by ACL reagent. The image used KODAK 1D 3.5 software to analyze, then normalize data to draw a graph by EXCEL.

According to implementations, human cytokine antibody arrays (C series 1000) were used to analyze cytokines according to the manufacturer's instructions (RayBiotech, Atlanta, Ga.). Briefly, the cell cultured condition medium was used to hybridize with the cytokine antibody array membrane, followed by addition of biotin-antibody cocktail. HRP-conjugated streptavidin was then used for detection of signals (enhanced chemiluminescence, ECL). The signal intensity of tested sample spots was determined by one-dimensional image densitometry analysis software (Kodak Scientific Imaging) and Raybio antibody array analysis tool (RayBiotech). The relative intensities of signals were normalized against the positive controls on each array membrane. Culture medium containing FBS was also used as a control experiment to provide basal signals and exclude possible cross-reactions in the cytokine array studies.

### Example 20

### Ex vivo Expansion of Hematopoietic Stem/Progenitor Cells

CD34+ cells were enriched from cord blood MNCs by magnetic activated cell sorting (MACs) with direct CD34 progenitor cell isolation kit (Miltenyi Biotech). The purity of the cell population was analyzed by flow cytometry. Briefly, isolated CD34+ cells were cultured at a density of 2×10⁴ cells/ml for expansion in IMDM (Gibco) containing 20% FBS (Hyclone) and a cytokine (R&D) cocktail of tong/ml rhTPO, 50 ng/ml rhSCF, 10 ng/ml rhIL-3, 10 ng/ml rhIL-6, and 50 ng/ml Flt-3 ligand. CD34+ HSCs with or without addition of F3 were cultured in a water-saturated atmosphere with 5% CO₂ for 7 days.

### Example 21

### Endothelium Tube Forming Assay

Endothelium cell tube formation assay was performed with Matrigel basement membrane matrix (BD Biosciences) according to the manufacture's instruction. Briefly, AD-MSCs derived endothelial cells were mixed with EGM-2 (Cambrex) media in the presence of 2 mM VEGF. Capillary tube-like formation was checked every 2 hours by phase contrast microscope.

### Example 22

### Real-Time PCR Array

The cultured cellular RNA was isolated using RNeasy® Total RNA isolation kit (Qiagen) and cDNA was synthesized using the SuperScript™ First-strand Synthesis System (Life Technologies). The cDNA was then added to the RT² SYBR Green qPCR Master Mix (SABiosciences). Real-time PCR was then performed on each sample using the RT² Profiler™ PCR Array kit for analysis of human angiogenesis and human tumor metastasis (SABiosciences). All real-time PCR reactions were done by ABI7300 system, with the following cycling conditions: an initial denaturation at 95° C. for 15 min, and 40 cycles of 95° C. for 15 s, 60° C. for 1 min. To analyze the real-time PCR array data, an MS-Excel sheet with macros was followed by the manufacturer's instruction at website.

1. A method comprising:
administering Ganoderma lucidum to a subject in an amount sufficient to reduce or prevent differentiation of the primitive CD34+ hematopoietic stem cells (HSCs).

2. The method of claim 1 wherein the CD34+ HSCs comprise one or more of the primitive hematopoietic cell population comprising CD38−, CD133+, CXCR4+, or Lin-markers.

3. A method comprising:
administering Ganoderma lucidum to a subject in an amount sufficient to stimulate blood mononuclear cells (MNCs) such that to influence the dendritic cell maturity.

4. The method of claim 3 wherein the dendritic cells comprise one or more of the group consisting of CD1a+, CD40+, CD80+, CD83+ and CD86+.

5. A method comprising:
administering Ganoderma lucidum to a subject in an amount sufficient to reduce or inhibit angiogenesis.

6. The method of claim 5 wherein the Ganoderma lucidum reduces or inhibits endothelial progenitor (EPCs) colony formation.

7. The method of claim 5 wherein the Ganoderma lucidum reduces or inhibits the expression of angiogenic factors including at least one of angiopoietins, Flt-1, Flt-4(VEGFR-3), KDR, PECAM(CD31), and leptin.

8. The method of claim 5 wherein the Ganoderma lucidum reduces or inhibits endoglin.

9. A method comprising:
administering Ganoderma lucidum to a subject in an amount sufficient to accelerate or enhance condrogenesis and chondrosphere formation of mesenchymal stem/stromal cells (MSCs).

10. The method of claim 9 further comprising increasing expression of one or more of the cell adhesion and interactive molecules, wherein the cell adhesion and interactive molecules comprise at least one of N-CAM, ICAM, IL-11, BMP-2, and aggrecan.
